(19) United States
(12) Patent Application Publication
(10) Pub. No.: US 2024/0307184 A1
(43) Pub. Date: Sep. 19, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Jesse Robert Edwards, Silverado, CA (US); Travis Zenyo Oba, Yorba Linda, CA (US); Andrew Charles May, Irvine, CA (US)

(21) Appl. No.: 18/662,753

(22) Filed: May 13, 2024

Related U.S. Application Data
(63) Continuation of application No. PCT/US2022/050158, filed on Nov. 16, 2022.
(60) Provisional application No. 63/281,587, filed on Nov. 19, 2021.

(51) Int. Cl. A61F 2/24

(52) U.S. Cl. CPC A61F 2/246; A61F 2/2454; A61F 2220/0008; A61F 2220/0091

(57) ABSTRACT

An implantable device includes one or more anchors. The anchors are configured to capture one or more leaflets of a native heart valve. The one or more anchors are configured to draw native valve leaflets into the device. The anchor portion can be extendable and retractable. The one or more anchors can be closed to secure the implantable device to the native valve leaflets.

### RELATED APPLICATIONS

The present application is a continuation of Patent Cooperation Treaty Application no. PCT/US2022/050158, filed on Nov. 16, 2022, which claims the benefit of U.S. Provisional Application No. 63/281,587, filed on Nov. 19, 2021, titled “HEART VALVE REPAIR DEVICES AND DELIVERY DEVICES THEREFOR”, which are incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation can have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation can be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure may be included in the examples summarized here.

An implantable device or implant (e.g., implantable prosthetic device, etc.) is configured to be positioned within a native heart valve to allow the native heart valve to form a more effective seal.

In some implementations, an implantable device includes one or more anchors. The anchors are configured to capture one or more leaflets of a native heart valve. The one or more anchors can be configured to draw native valve leaflets into the device. The anchor portion can be extendable and retractable. The one or more anchors can be closed to secure the implantable device to the native valve leaflets.

In some implementations, an implantable device includes a capture element and an anchor portion. The anchor portion includes one or more anchors. The anchor portion is disposed at least partially in the capture element. The anchors are configured to capture one or more leaflets of a native heart valve. The anchor portion is extendable out of the capture element and retractable into the capture element. The capture element and the anchor portion are configured to draw native valve leaflet tissue into the capture element when the anchor portion is retracted into the capture element.

In some implementations, the capture element includes an inner cavity from which the anchor portion is extendable out of and into which the native valve leaflet tissue can be drawn into.

In some implementations, an implantable device or implant includes an anchor portion having an inner anchor body and an outer anchor body. The inner anchor body can include one or more anchors configured to attach to one or more leaflets of a native heart valve.

In some implementations, the capture element is cylindrical in shape and/or is round in cross section. In some implementations, the inner cavity extends from a first end of the capture element to a second end of the capture element.

In some implementations, in a closed position, the anchor portion is housed entirely within the inner cavity. In some implementations, in an open position, the anchor portion is housed at least partially outside of the inner cavity.

In some implementations, the capture element is impervious to blood. In some implementations, the capture device inhibits or reduces blood flow.

In some implementations, the capture element comprises an opening in an end wall at the first end of the capture element. In some implementations, the second end of the capture element is open, such that the anchor portion can be moved in and out of the capture element from the second end. In some implementations, the capture element comprises a one-way valve.

In some implementations, the one or more anchors are made from a flexible or expandable material. In some implementations, the anchor portion comprises a body coupled to the one or more anchors. In some implementations, the anchor portion is expandable.

In some implementations, the capture element is removably attached to a delivery catheter. In some implementations, the anchor portion is removably attached to an actuation element. In some implementations, the actuation element is disposed radially inward of the delivery catheter.

In some implementations, the actuation element is attached to a collar of the anchor portion.

Movement of the actuation element, in some implementations, can move the anchor portion between a closed position and an open position.

In some implementations, the actuation element is connected to the anchor portion such that a user can provide a tensioning force to the actuation element to cause the anchor portion to move from an expanded position having an expanded width to a narrowed position having a narrowed width, wherein the expanded width is greater than the narrowed width.

In some implementations, an implantable device includes a capture element and an anchor portion. In some implementations, the anchor portion includes an inner anchor body and an outer anchor body.

In some implementations, the anchor portion is disposed at least partially in the capture element, and the capture element includes an inner cavity.

In some implementations, the outer anchor body is extendable out of the inner cavity and retractable into the inner cavity. In some implementations, the inner anchor body is extendable out of the outer anchor body and retractable into the outer anchor body.

In some implementations, the inner anchor body and the outer anchor body are configured to capture native valve leaflet tissue therebetween.

In some implementations, the capture element and the anchor portion are configured to draw the native valve leaflet tissue into the inner cavity when the anchor portion is retracted into the inner cavity of the capture element.

In some implementations, the capture element is cylindrical in shape and/or is round in cross section. In some implementations, the inner cavity extends from the first end of the capture element to the second end of the capture element.

In some implementations, in a closed position, the anchor portion is housed entirely within the inner cavity. In some implementations, in an open position, the anchor portion is housed at least partially outside of the inner cavity.

In some implementations, the capture element is impervious to blood. In some implementations the capture element inhibits or reduces blood flow.

In some implementations, the capture element comprises an opening in the first end of the capture element. In some implementations, the second end of the capture element is open, such that the anchor portion can be moved in and out of the capture element from the second end. In some implementations, the capture element comprises a one-way valve.

In some implementations, the anchor portion is made from a flexible or expandable material. In some implementations, the anchor portion is expandable.

In some implementations, the capture element is removably attached to a delivery catheter. In some implementations, the anchor portion is removably attached to one or more actuation elements.

In some implementations, the inner anchor body is removably attached to an inner actuation element. In some implementations, the outer anchor body is removably attached to an outer actuation element.

In some implementations, the one or more actuation elements are disposed radially inward of the delivery catheter. In some implementations, movement of the inner actuation element can move the inner anchor body between a closed position and an open position.

In some implementations, movement of the outer actuation element can move the outer anchor body between a closed position and an open position.

In some implementations, the one or more actuation elements is connected to the anchor portion such that a user can provide a tensioning force to the one or more actuation elements to cause the anchor portion to move from an expanded position having an expanded width to a narrowed position having a narrowed width, wherein the expanded width is greater than the narrowed width.

In some implementations, a method of repairing a native valve includes positioning anchor portions such that leaflets of a native heart valve are disposed in the anchor portions and drawing the anchor portions and portions of the leaflets into an open end of a capture element. In some implementations, the capture element has a first end, a second end, and a cavity between the first end and the second end.

In some implementations, the anchor portions are extendable out of the cavity of the capture element and retractable into the cavity of the capture element.

In some implementations, the method includes blocking blood flow with the capture element. In some implementations, the method further includes decoupling the anchor portions and the capture element from a delivery catheter.

The above method(s) can be performed on a living animal or on a simulation, such as on a cadaver, cadaver heart, anthropomorphic ghost, simulator (e.g., with body parts, heart, tissue, etc. being simulated).

In some implementations, an implantable device includes an anchor portion and a rotating member. In some implementations, the anchor portion includes one or more anchors. In some implementations, the anchor portion is coupled with a capture element.

In some implementations, the rotating member is coupled with the one or more anchors and is configured to draw native valve leaflet tissue into the one or more anchors.

In some implementations, the rotating member is cylindrical. In some implementations, the rotating member includes one or more projections, notches, or other gripping members spaced throughout the rotating member for coupling with leaflets.

In some implementations, the rotating member include a ridge that is threaded along a surface of the rotating member. In some implementations, the rotating member is removably attached to an actuation element.

In some implementations, in response to the rotating member rotating in a first direction, the rotating member draws a leaflet into the one or more anchors. and in response to the rotating member rotating in a second direction, the rotating member moves the leaflet out of the one or more anchors.

In some implementations, a device (e.g., a valve repair device, an implant, etc.) is adapted to be implanted between leaflets of a native heart valve. The device includes a gripping member and a leaflet repositioning device.

In some implementations, the gripping member including a base arm and a moveable arm and is configured to move between an open position and a closed position. In the closed position, the gripping member is configured to grasp a leaflet of a native heart valve between the base arm and the movable arm.

In some implementations, the leaflet repositioning device is configured to reposition the leaflet relative to the base arm while the gripping member is in the closed position.

In some implementations, the leaflet repositioning device is configured to move the movable arm, relative to the base arm, toward a centerline of the device while the gripping member is in the closed position.

In some implementations, the movable arm has a proximal end and a distal end, and the leaflet repositioning device includes a retraction element attached to the proximal end, wherein pulling the retraction element while the gripping member is in the closed position causes the distal end to move toward a centerline of the device.

In some implementations, the device further includes coaptation element positioned along the centerline of the device, wherein pulling the retraction element causes the movable arm to retract into the coaptation element.

In some implementations, the device further includes a lock configured to secure the movable arm in position after being moved by the leaflet repositioning device.

In some implementations, the device further includes a securing element disposed on the movable arm for engaging the leaflet. In some implementations, the securing element includes one or more barbs. In some implementations, the device further includes a second securing element disposed on the base arm for engaging the leaflet.

In some implementations, the leaflet repositioning device is configured to move the securing element, relative to the movable arm, toward a centerline of the device while the gripping member is in the closed position.

In some implementations, the movable arm has a proximal end and a distal end, and the leaflet repositioning device includes a retraction element attached to the securing element end. Pulling the retraction element while the gripping member is in the closed position causes the securing element to move toward a centerline of the device.

In some implementations, the device further includes a coaptation element positioned along the centerline of the device.

In some implementations, the device further includes a lock configured to secure the securing element in position relative to the movable arm after being moved by the leaflet repositioning device.

In some implementations, the securing element includes one or more barbs. In some implementations, the securing element has a distal position and a proximal position that is inward of the distal position.

In some implementations, the leaflet repositioning device can be configured to move the securing element repeatedly between the distal position and proximal position to incrementally move the leaflet toward the centerline of the device.

In some implementations, the leaflet repositioning device includes a ratcheting device operable to move the securing element repeatedly between the distal position and proximal position.

In some implementations, the device includes a second securing element disposed on the base arm for engaging the leaflet. In some implementations, the securing element disposed on the movable arm can move from the proximal position to the distal position while the second securing element holds the leaflet in position relative to the movable arm.

In some implementations, the leaflet repositioning device is configured to rotate the movable arm, relative to the base arm, about a longitudinal axis of the movable arm while the gripping member is in the closed position. In some implementations, rotating the movable arm moves the leaflet relative to the base arm.

In some implementations, the device includes a lock configured to lock the movable arm in rotational position after being moved by the leaflet repositioning device.

In some implementations, the device includes a securing element disposed on the movable arm for engaging the leaflet. In some implementations, the securing element includes one or more barbs.

In some implementations, the device further includes a second gripping member including a second base arm and a second moveable arm. In some implementations, the second gripping member is configured to move between an open position and a closed position. In the closed position, the second gripping member is configured to grasp a second leaflet of a native heart valve between the second base arm and the second movable arm.

In some implementations, the leaflet repositioning device is configured to rotate the second movable arm, relative to the second base arm, about a second longitudinal axis of the second movable arm while the second gripping member is in the closed position.

In some implementations, the device includes a second leaflet repositioning device. In some implementations, the second repositioning device is configured to rotate the second movable arm, relative to the second base arm, about a second longitudinal axis of the second movable arm while the second gripping member is in the closed position. In some implementations, the second movable arm and the movable arm are configured to rotate simultaneously in opposite directions.

In some implementations, a method of repairing a native valve includes delivering an implantable device to the native valve, positioning the implantable device in annulus of the native valve, closing a gripping member of the implantable device to grasp a leaflet of the native valve, and repositioning the leaflet relative to the base arm while the gripping member remains closed.

In some implementations, the gripping member has a base arm and a movable arm. In some implementations, repositioning the leaflet includes moving the movable arm, relative to the base arm, toward a centerline of the device.

In some implementations, moving the movable arm includes applying tension to a retraction element coupled to the movable arm. In some implementations, applying tension to the retraction element includes retracting the movable arm into a coaptation element of the device.

In some implementations, the method includes locking the movable arm in position after repositioning the leaflet.

In some implementations, closing the gripping member includes engaging the leaflet with a securing element disposed on the movable arm. In some implementations, the securing element includes one or more barbs.

In some implementations, closing the gripping member includes engaging the leaflet with a second securing element disposed on the base arm.

In some implementations, the securing element is disposed on the movable arm and repositioning the leaflet includes moving the securing element, relative to the movable arm, toward a centerline of the device while the gripping member is in the closed position.

In some implementations, moving the securing element includes applying tension to a retraction element coupled to the securing element.

In some implementations, the method includes locking the securing element in position relative to the movable arm after being moved by the leaflet repositioning device. In some implementations, the securing element includes one or more barbs.

In some implementations, the securing element has a distal position and a proximal position that is inward of the distal position.

In some implementations, repositioning the leaflet includes repeatedly moving the securing element between the distal position and proximal position to incrementally move the leaflet toward the centerline of the device.

In some implementations, a second securing element is disposed on the base arm for engaging the leaflet.

In some implementations, repeatedly moving the securing element between the distal position and proximal position includes holding the leaflet in position relative to the movable arm with the second securing element when the securing element disposed on the movable arm moves from the proximal position to the distal position.

In some implementations, repositioning the leaflet includes rotating the movable arm, relative to the base arm, about a longitudinal axis of the movable arm while the gripping member is in the closed position.

In some implementations, rotating the movable arm moves the leaflet relative to the base arm. In some implementations, the method includes locking the movable arm in a rotational position after repositioning the leaflet.

In some implementations, closing the gripping member includes engaging the leaflet with a securing element disposed on the movable arm. In some implementations, the securing element includes one or more barbs.

In some implementations, the method includes closing a second gripping member of the implantable device to grasp a second leaflet of the native valve.

In some implementations, the second gripping member includes a second base arm and a second movable arm. In some implementations, the method includes repositioning the second leaflet relative to the second base arm while the second gripping member remains closed.

In some implementations, repositioning the second leaflet includes rotating the second movable arm, relative to the second base arm, about a second longitudinal axis of the second movable arm while the second gripping member is in the closed position.

In some implementations, rotating the second movable arm and rotating the first movable arm are done simultaneously in opposite directions.

The above method(s) can be performed on a living animal or on a simulation, such as on a cadaver, cadaver heart, anthropomorphic ghost, simulator (e.g., with body parts, heart, tissue, etc. being simulated).

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 illustrates a healthy mitral valve with the leaflets closed as viewed from an atrial side of the mitral valve;

FIG. 5 illustrates a dysfunctional mitral valve with a visible gap between the leaflets as viewed from an atrial side of the mitral valve;

FIG. 6 illustrates a tricuspid valve viewed from an atrial side of the tricuspid valve;

FIG. 7 illustrates an exploded view of a device or implant;

FIGS. 8-9 illustrate an example of a device or implant;

FIGS. 10-14 show an example of a device or implant, in various stages of deployment;

FIG. 15 illustrates a top view of a mitral valve with a device or implant in use;

FIG. 16 illustrates an example of a device or implant;

FIG. 17 illustrates an exploded view of the device or implant of FIG. 16;

FIG. 18 illustrate the device or implant of FIG. 16 coupled to a delivery system;

FIGS. 19-23 show the device or implant of FIG. 16 in various stages of deployment;

FIGS. 24-29 show the example device or implant of FIGS. 7-14 being delivered and implanted within a native valve;

FIG. 30 illustrates an example delivery system to deliver components of a valve prosthesis;

FIG. 31 illustrates an example of a valve prosthesis in an expanded configuration;

FIG. 32 illustrates an example of a device or implant,

FIG. 33 illustrates an example of a device or implant; and

FIG. 34 illustrates an example of a device or implant.

FIG. 35 illustrates an example of a device or implant.

FIG. 36-38 illustrate the example device or implant of FIG. 35, including a cover, at various stages of deployment within a native valve.

FIG. 39 illustrates an example of a device or implant.

FIG. 40-42 illustrates the example device or implant of FIG. 39 at various stages of deployment within a native valve.

FIG. 43 illustrates an example of a device or implant.

FIG. 44-45 illustrates adjustment of a position of the example device or implant of FIG. 43 in a native valve.

### DETAILED DESCRIPTION

“The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

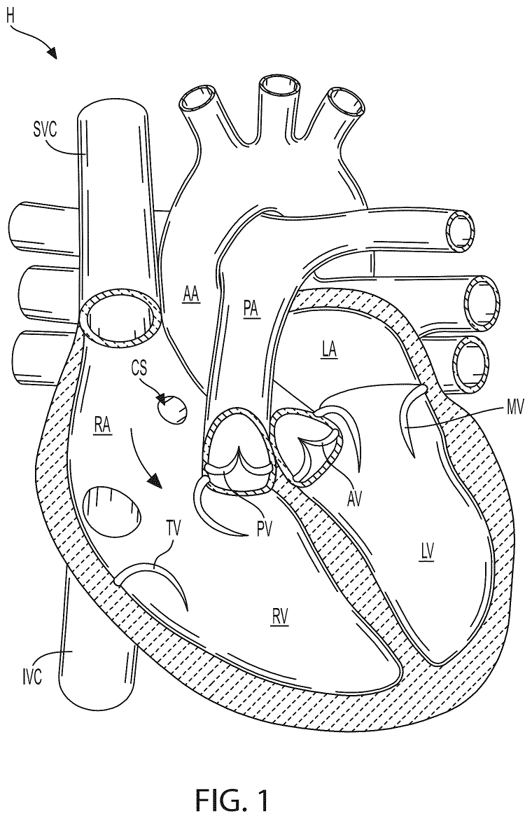

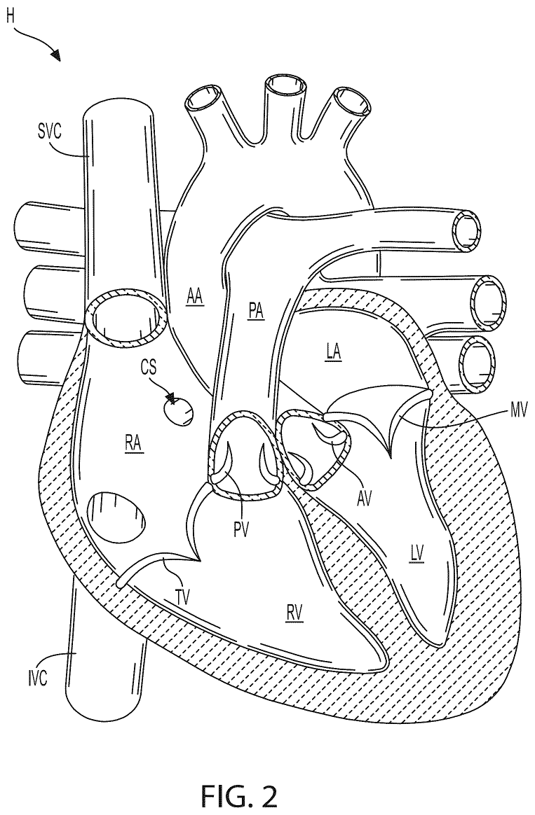

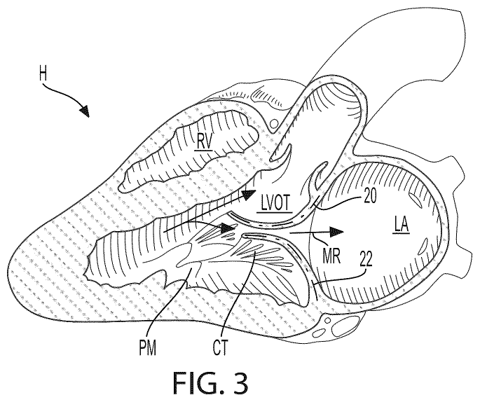

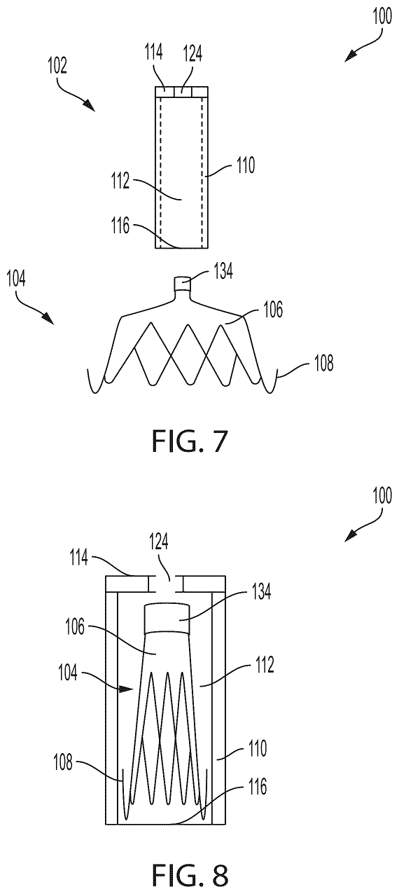

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit, or reduce blood from regurgitating from the left ventricle LV and back into the left atrium LA. Many of the devices described in the present application are designed to easily grasp and secure the native leaflets to prevent or inhibit back flow or regurgitation during systole, though this is not necessary.

Referring now to FIGS. 1-6, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroblastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease or dilation of a ventricle.

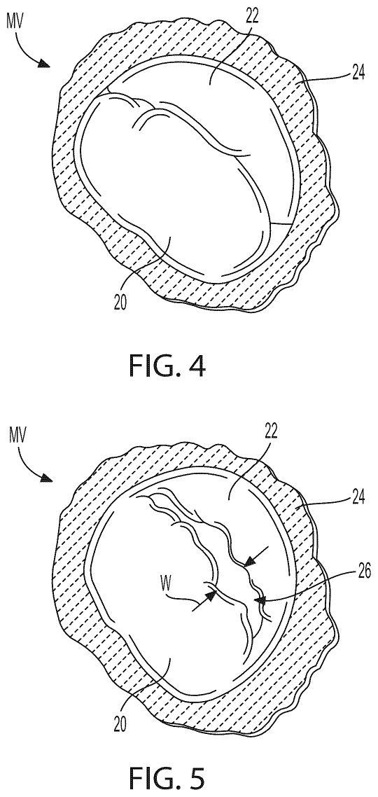

Referring to FIG. 4, when a healthy mitral valve MV is in a closed position, the anterior leaflet 20 and the posterior leaflet 22 coapt, which prevents blood from leaking from the left ventricle LV to the left atrium LA. Referring to FIGS. 3 and 4, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 4. Referring to FIG. 5, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV.

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) is primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

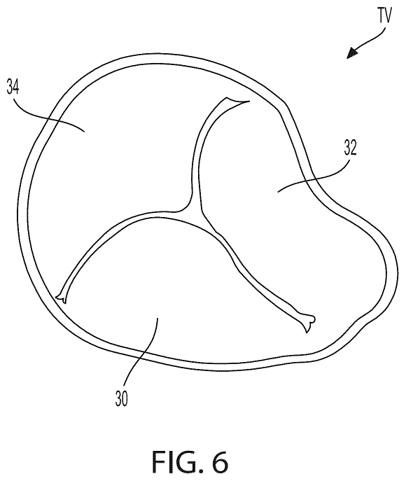

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 6), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

The disclosed devices or implants can be configured such that an anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

Referring to FIGS. 7-9, an implantable device 100 (e.g., an implant, a prosthetic device, a prosthetic spacer device, valve repair device, etc.) is illustrated. Other similar devices/implants are described in more detail in PCT patent application publication Nos. WO2018/195215, WO2020/076898, and WO 2019/139904, which are incorporated herein by reference in their entireties. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

With reference to FIG. 7, the device 100 is illustrated and includes a capture portion 102 and an anchor portion 104. The device 100 is configured to be positioned within the native heart valve orifice between the leaflets, thereby reducing or preventing regurgitation described above. The device 100 can be configured to attach to and/or seal against two or three native valve leaflets. The device 100 can be used in the native mitral (bicuspid) and tricuspid valves.

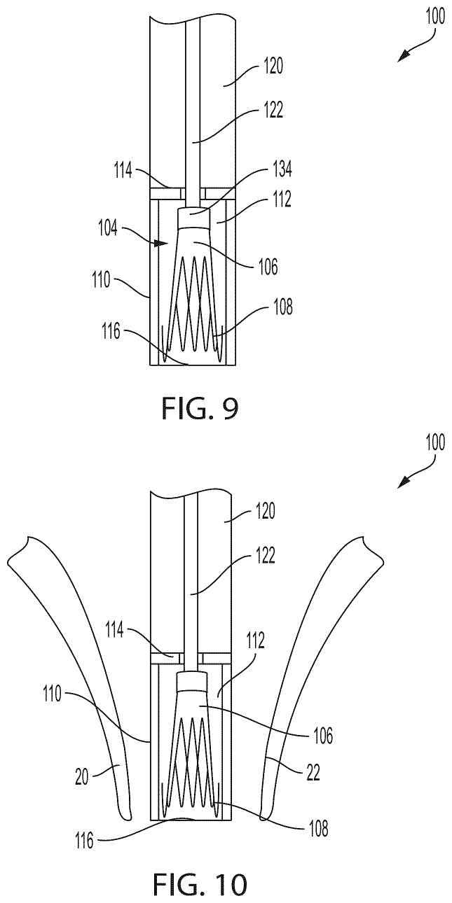

The capture portion 102 includes a capture element 110. In some implementations, the capture element 110 is adapted to be implanted between leaflets of a native valve (e.g., a native mitral valve, native tricuspid valve, etc.) and is attached to an actuation element (e.g., actuation wire, actuation shaft, actuation tube, actuation rod, etc.) or a delivery system 120. The delivery system can comprise one or more of a guide/delivery sheath, a delivery catheter, a steerable catheter, an implant catheter, tube, combinations of these, etc. With reference to FIG. 9, the capture element 110 can be removably coupled to a catheter of the delivery system 120.

With reference to FIG. 7, the capture element 110 can have various shapes. In some implementations, the capture element can have an elongated cylindrical shape having a round cross-sectional shape. In some implementations, the capture element can have an oval cross-sectional shape, an ovoid cross-sectional shape, a crescent cross-sectional shape, a rectangular cross-sectional shape, or various other non-cylindrical shapes. The capture element includes an inner cavity 112 that extends from a wall at a first end 114 of the capture element 110 to an open second end 116 of the capture element 110. In some implementations, the wall at the first end 114 of the capture element 110 includes an opening 124 at the first end of the capture element 110. In some implementations, the opening of the second end 116 of the capture element 110 is configured, such that the anchor portion 104 can be moved in and out of the capture element 110.

The capture element 110 can optionally have a structure that is impervious to blood (or that resists blood flow therethrough) and that allows the native leaflets to close around the capture element during ventricular systole to block blood from flowing from the left or right ventricle back into the left or right atrium, respectively. In some implementations, the capture element 110 can optionally include a one-way valve through the inner cavity 112 and out the opening 124, such that a fluid (e.g., blood) can travel out of the opening 124 from the cavity 112, but not into the cavity from the opening 124.

The anchor portion 104 includes a body 106 and one or more anchors 108. The body 106 and anchors 108 can comprise a variety of shapes and can be made from a variety of materials or substances. For example, the body 106 and/or anchors 108 can be made from a flexible or expandable material.

The anchors 108 can take a wide variety of forms, such as, for example, paddles, gripping elements, or the like. The anchors can be jointed and/or flexible. The anchors 108 can be curved or rounded such that a leaflet can fit into the curve and be secured by the anchor 108. In some implementations, the anchors can include attachment portions or gripping members. The illustrated gripping members can comprise clasps, optional barbs, friction-enhancing elements, or other means for securing (e.g., protrusions, ridges, grooves, textured surfaces, adhesive, etc.).

With reference to FIG. 8, the anchor portion 104 can be entirely housed within the inner cavity 112 of the capture element 110 such that the anchor body 106 and anchors 108 are disposed between the first end 114 and the second end 116 of the capture element 110. The anchor portion 104, when housed in the inner cavity 112 of the capture element 110 is in a closed position. When the device 100 is in a closed position (with and/or without valve leaflet tissue), the anchor portion 104 can be secured within the inner cavity 112 of the capture element 110, for example, by a friction fit.

FIG. 9 illustrates the device 100 coupled to a delivery system. The device 100 can be configured to be implanted via a delivery system or other means for delivery. The capture element 110 can be coupled to the catheter of the delivery system 120 in a variety of ways, including a selectively releasable coupling, a press fit, a friction fit, a magnetic fit, by mating threads, etc.

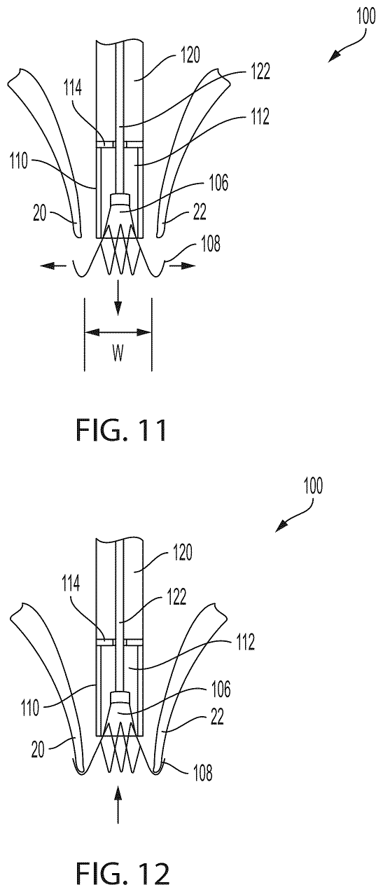

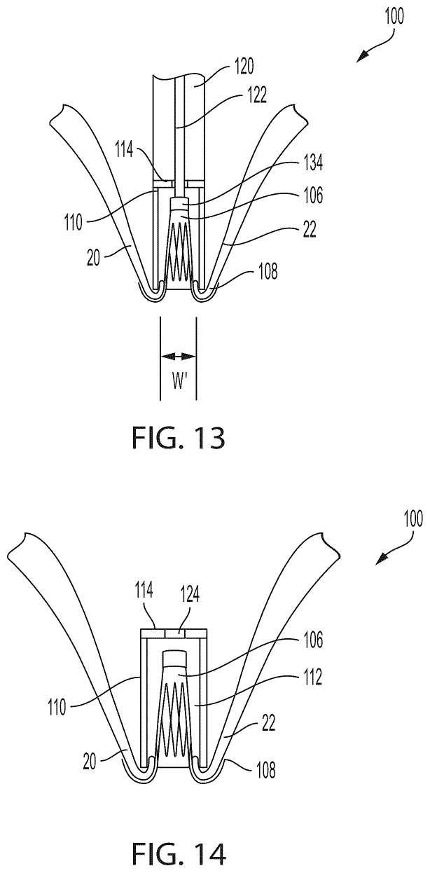

The anchor portion 104 can be coupled to an actuation element 122. The actuation element 122 can take a wide variety of different forms, including a wire, rod, shaft, tube, screw, suture, line, strip, or a combination of these. The actuation element 122 can be made of a variety of different materials and have a variety of configurations. As one example, the actuation element can be threaded such that rotation of the actuation element moves the anchor portion relative to the capture element. Or, the actuation element can be unthreaded, such that pushing or pulling the actuation element moves the anchor portion relative to the capture element 110. The actuation element 122 can be disposed through an opening 124 in the first end 114 of the capture element 110 and attach to the anchor portion 104. In some implementations, the actuation element 122 can attach to the body 106 or a collar 134 of the body 106. The actuation element 122 can be connected to the anchor portion 104 such that a user can provide a force to the actuation element 122 to cause the anchor portion 104 to move from an expanded position (FIG. 11) having an expanded width W to a narrowed position (FIG. 13) having a narrowed width W′, wherein the expanded width W is greater than the narrowed width W′.

With reference to FIGS. 10-14, the device 100 can be positioned in a heart valve between opposing leaflets. In some implementations, the anchor 108 and the capture element 110 can be positioned simultaneously by moving the anchor 108 and the capture element 110 together along the longitudinal axis of the actuation element 122. The anchor 108 can be configured to be positioned behind a native leaflet when implanted such that the leaflet is grasped by the anchor 108.

With reference to FIG. 11, the device 100 can be configured for the anchor portion 104 to be moved along the longitudinal axis of the actuation element 122 away from the capture element 110 in order to create a gap between the capture element 110 and the anchors 108. The movement of the actuation element 122 can push the anchors 108 out of the capture element 110 and into the ventricular or lower portion of the heart. In some implementations, the body 106 is configured to self-expand as it moves distally out from the capture element 110.

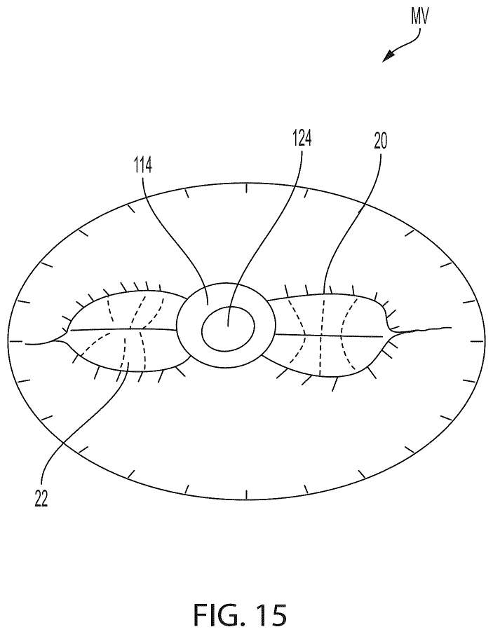

With reference to FIG. 12, the device 100 can be moved such that the native leaflets (e.g., mitral leaflets 20, 22) are positioned in the gap between the anchors 108 and the capture element 110. In some implementations, the actuation element 122 is moved proximally and the anchors 108 are pulled towards the capture element 110 to secure the leaflets 20, 22 within the anchors 108. As the anchors 108 secure the leaflets 20, 22, the actuation element 122 and anchor portion 104 are pulled further towards the capture element 110 until the leaflets 20, 22 are secured against the capture element 110 and the anchors 108 (FIG. 13). In the example illustrated by FIG. 13 portions of the leaflets 20, 22 are pulled inside the capture element. A portion of the leaflet can be pressed between the inside surface of the capture element and the anchors, between an end surface of the capture element and the anchors, and/or between the outside surface of the capture element and the anchors. In the example illustrated by FIGS. 13 and 14, a portion of the leaflet is pressed between all of the inside surface of the capture element and the anchors, the end surface of the capture element and the anchors, and the outside surface of the capture element and the anchors. Once the leaflets 20, 22 are pressed between the capture element and the anchors 108, the actuation element 122 and delivery system 120 can be decoupled from the device 100 as illustrated by FIG. 14.

In some implementations, the capture element 110 can be moved towards the anchors 108, thereby closing the gap between the capture element 110 and the anchors 108 and capturing the leaflets 20, 22 between the capture element 110 and the anchors 108.

With reference to FIG. 15, the anchor can be configured to secure the device to one or both of the leaflets 20, 22 such that the capture element 110 is positioned between the leaflets 20, 22. In some implementations configured for use in the tricuspid valve, the anchor is configured to secure the device to one, two, or three of the tricuspid leaflets such that the capture element is positioned between two of the native leaflets or all three native leaflets.

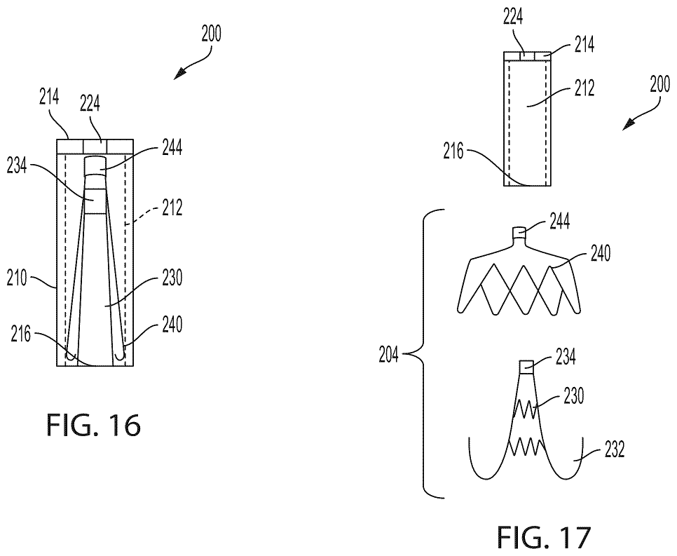

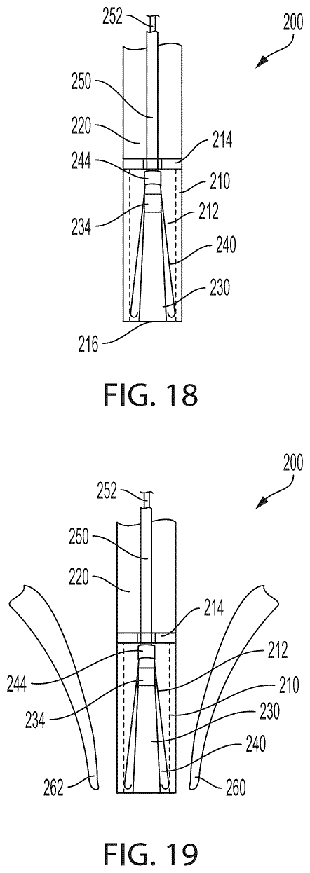

With reference to FIGS. 16-17, an example device 200 is illustrated. The device 200 includes a capture element 210 and an anchor portion 204. The capture element 210 can be the same or similar to the capture element 110 (FIGS. 7-14) in all material aspects. The capture element 110 is adapted to be implanted between leaflets of a native valve (e.g., a native mitral valve, native tricuspid valve, etc.) and is attached to an actuation element (e.g., actuation wire, actuation shaft, actuation tube, actuation rod, etc.) or the catheter 220 of the delivery system (FIGS. 18-22). The capture element 210 can have an elongated cylindrical shape having a round cross-sectional shape or another cross-sectional shape. In some implementations, the shape of the capture element has the same shape as the annulus of the native mitral valve but is scaled down. The capture element comprises an inner cavity 212 that extends from a first end 214 of the capture element 210 to a second end 216 of the capture element 210.

The capture element 210 can have a structure that is impervious to blood (or that resists blood flow therethrough) and that allows the native leaflets to close around the capture element during ventricular systole to block blood from flowing from the left or right ventricle back into the left or right atrium, respectively. The capture element can include a check valve that allows blood to flow from the first end 214 to the second end 216, but blocks blood flow in the direction from the second end 216 to the first end. In some implementations, the optional check is disposed in the opening 224.

The anchor portion 204 of the device 200 includes an inner anchor body 230 and an outer anchor body 240. The inner anchor body 230 and the outer anchor body 240 can comprise a variety of shapes and be made from a variety of materials or substances. For example, the inner anchor body 230 and/or the outer anchor body 240 can be made from a flexible and/or expandable material.

The inner anchor body 230 and the outer anchor body 240 can be configured to expand and contract. For example, the inner anchor body 230 and the outer anchor body 240 can contract or be compressed to fit within the cavity 212 of the capture element 210. In various implementations when the inner anchor body 230 and the outer anchor body 240 are removed from the cavity 212 of the capture element 210, the inner anchor body 230 and the outer anchor body 240 are configured to expand. For example, the inner anchor body 230 and/or the outer anchor body 240 can have a stent or stent-like configuration with struts that allow expansion and contraction.

Referring to FIG. 17, the inner anchor body 230 can include one or more anchors 232. The anchors 232 can take a wide variety of forms, such as, for example, hooks, paddles, gripping elements, or the like. The anchors can be jointed and/or flexible. The anchors 232 can be curved or rounded such that a leaflet can fit into the curve and be secured by the anchor 232. In some implementations, the anchors can include attachment portions or gripping members. The illustrated gripping members can comprise clasps, optional barbs, friction-enhancing elements, or other means for securing (e.g., protrusions, ridges, grooves, textured surfaces, adhesive, etc.).

With reference to FIG. 16, the anchor portion 204 can be entirely housed within the inner cavity 212 of the capture element 210 such that the inner anchor body 230 and the outer anchor body 240 are disposed between the first end 214 and the second end 216 of the capture element 210. The anchor portion 204, when housed in the inner cavity 212 of the capture element 110 is in a closed position.

Referring to FIG. 18, the device 200 can be configured to be implanted via a delivery system or other means for delivery. The delivery system can comprise one or more of a guide/delivery sheath, a delivery catheter, a steerable catheter, an implant catheter, tube, combinations of these, etc. With reference to FIG. 18-22, the capture element 210 can be removably coupled to a catheter 220. The capture element 210 can be coupled to the catheter 220 in a variety of ways, including a releasable coupler, a releasable press fit, friction fit, magnetic fit, a threaded connection, etc.

The anchor portion 204 can be coupled to one or more actuation elements. In the illustrated example, the outer anchor body 240 is removably coupled to an outer actuation element 250. The outer actuation element 250 can be disposed radially inward of catheter 220. The outer actuation element 250 can be slidable relative to the catheter 220. In some implementations, the outer actuation element 250 can attach to the outer anchor body 240 at an outer collar 244.

The inner anchor body 230 is removably coupled to an inner actuation element 252. The inner actuation element 252 can be disposed radially inward of the outer actuation element 250. The inner actuation element 252 can be slidable relative to the outer actuation element 250. In some implementations, the inner actuation element 252 can attach to the inner anchor body 230 at an inner collar 234. The inner actuation element 252, outer actuation element 250, and the catheter 220 can all be moved simultaneously and independently of one another.

The outer actuation element 250 and inner actuation element 252 can take a wide variety of different forms, including a wire, rod, shaft, tube, screw, suture, line, strip, or a combination of these. The outer actuation element 250 and inner actuation element 252 can be made of a variety of different materials and have a variety of configurations. As one example, the actuation elements can be threaded such that rotation of the actuation element moves the anchor portion relative to the capture element. Or, the actuation elements can be unthreaded, such that pushing or pulling the actuation element moves the anchor portion relative to the capture element. The outer actuation element 250 and inner actuation element 252 can be disposed through the opening 224 in the first end 214 of the capture element 210.

With reference to FIGS. 19-23, the device 200 can be positioned in a heart valve between opposing leaflets. In some implementations, the anchor portion 204 and the capture element 210 can be positioned simultaneously by moving the anchor portion 204 and the capture element 210 together along the longitudinal axis of the catheter 220. The anchor portion 204 can be configured to be positioned behind a native leaflet when implanted such that one or more native leaflets (e.g., mitral leaflets 260, 262) are grasped by the anchor portion 204.

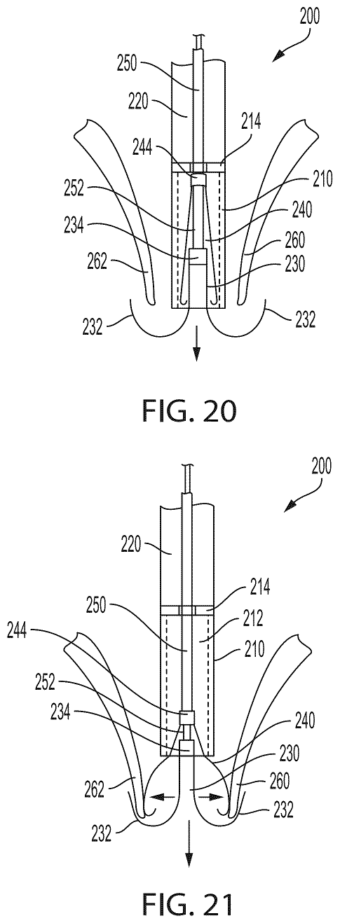

With reference to FIG. 20, the device 200 can be configured for the inner anchor body 230 to be moved along the longitudinal axis of the inner actuation element 252 away from the capture element 210 in order to create a gap between the capture element 210 and the anchors 232. In the illustrated example, the inner anchor body 230 is pushed out of the outer anchor body 240 and the capture element 210. The movement of the inner actuation element 252 can push the anchors 232 out of the capture element 210 and into the ventricular or lower portion of the heart. In some implementations, the inner anchor body 230 and anchors 232 are configured to self-expand as they move distally out from the capture element 210. For example, the anchor body 230 can have a self-expanding stent or stent-like configuration.

With reference to FIG. 21, the device 200 can be configured for the outer anchor body 240 to be moved along the longitudinal axis of the outer actuation element 250 away from the capture element 210. The movement of the outer actuation element 250 can push the outer anchor body 240 out of the capture element 210 and towards the leaflets 260, 262. As a result, the leaflets 260, 262 can be captured between the outer anchor body 240 and the inner anchor body 230. In the illustrated example, the anchors 232 expand radially outward further than the outer anchor body 240, such that the anchors 232 are disposed on an outer or ventricular side of the leaflets 260, 262 and the outer anchor body 240 is disposed on an inner or atrial side of the leaflets 260, 262. In some implementations, the outer anchor body 240 is configured to self-expand as it moves distally out from the capture element 210.

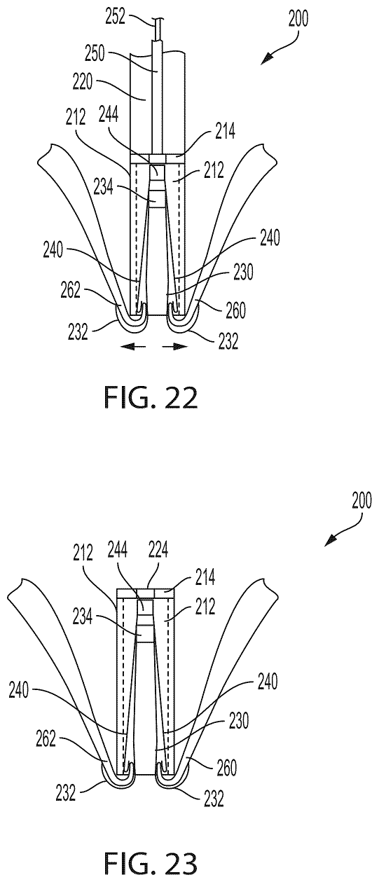

With reference to FIG. 21, leaflets 260, 262 can then be positioned between the outer anchor body 240 and the anchors 232. In some implementations, the inner actuation element 252 and outer actuation element 250 are moved proximally towards the capture element 210. The outer anchor body 240 and anchors 232 are pulled towards the capture element 210 to secure the leaflets 260, 262. As the inner actuation element 252 and outer actuation element 250 are pulled further proximally, the leaflets 260, 262 are secured against the outer anchor body 240, the capture element 210, and the anchors 232 (FIG. 22). With reference to FIG. 23, the actuation elements 250, 252 and delivery system or catheter 220 can be decoupled from the device 200, leaving the device attached to the native valve leaflets 260, 262.

In some implementations, the capture element 210 can be moved towards the anchors 232, thereby closing the gap between the capture element 210 and the anchors 232. Portions of the leaflets 260, 262 can be captured between the outer anchor body 240 and the inner anchor body 230 and/or between the capture element 210 and the anchors 232. Portions of the leaflets 260, 262 can be captured between an inside surface of the capture element 210 and the anchors 232, between an end surface of the capture element 210 and the anchors 232, between an outside surface of the capture element 210 and the anchors 232, and/or between the outer anchor body 240 and the anchors 232. In the example illustrated by FIG. 22, leaflet portions are captured between the outer anchor body 240 and the anchors 232, between an end surface of the capture element 210 and the anchors 232, and between an outside surface of the capture element 210 and the anchors 232. In some implementations configured for use in the tricuspid valve, the anchor is configured to secure the device to one, two, or three of the tricuspid leaflets such that the capture element is positioned between two of the native leaflets or between all three native leaflets.

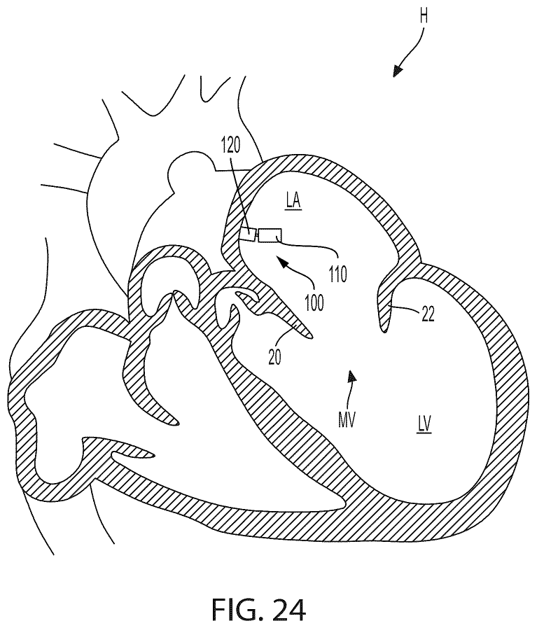

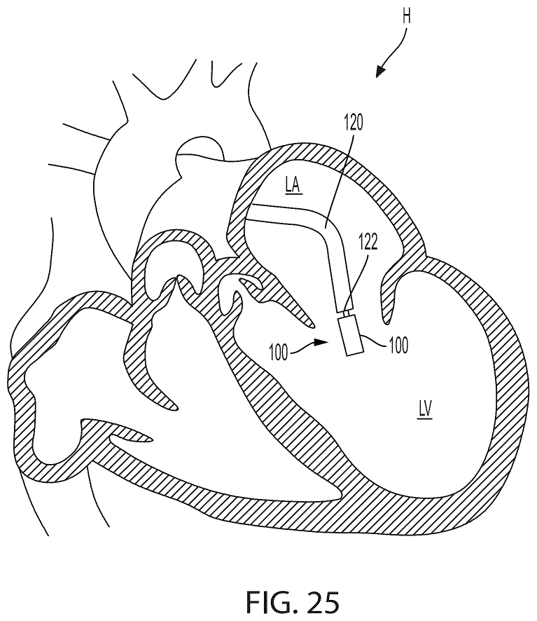

Referring now to FIGS. 24-29, the implantable device 100 of FIGS. 7-15 is shown being delivered and implanted within the native mitral valve MV of the heart H. The implantable device of FIGS. 16-23 can be delivered in the same manner, except the inner and outer anchor bodies 230, 240 are deployed as shown in FIGS. 20 and 21. Referring to FIGS. 24-25, a delivery sheath and/or catheter of the delivery system 120 is inserted into the left atrium LA through the septum and into the left ventricle.

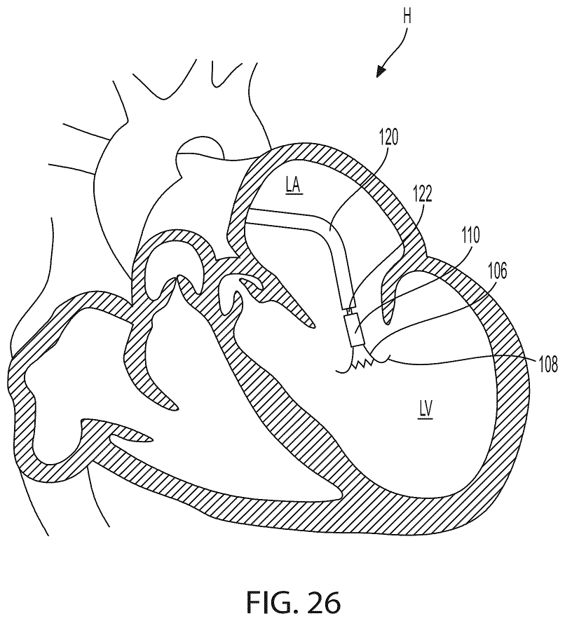

As can be seen in FIG. 26, the implantable device or implant 100 is moved into position within the mitral valve MV, into the ventricle LV and partially opened so that the leaflets 20, 22 can be grasped. For example, the catheter of the delivery system 120 can be advanced and steered or flexed to position the device 100 as illustrated by FIG. 26. The actuation element 122 can be advanced from inside the catheter of the delivery system 120 to position the implant outside the capture element 110 as illustrated by FIG. 26.

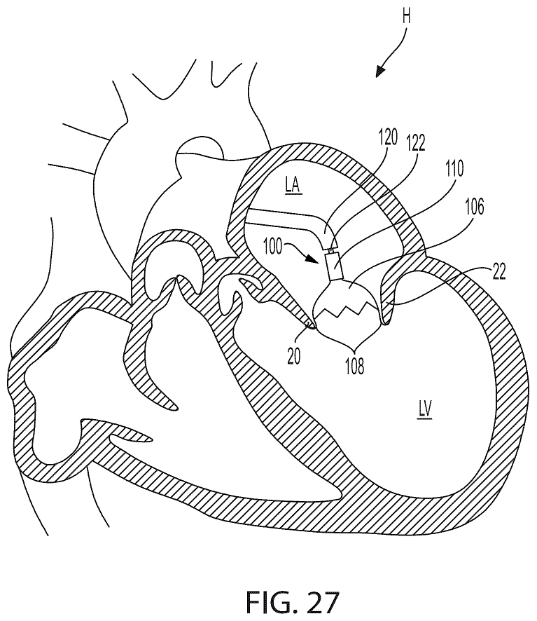

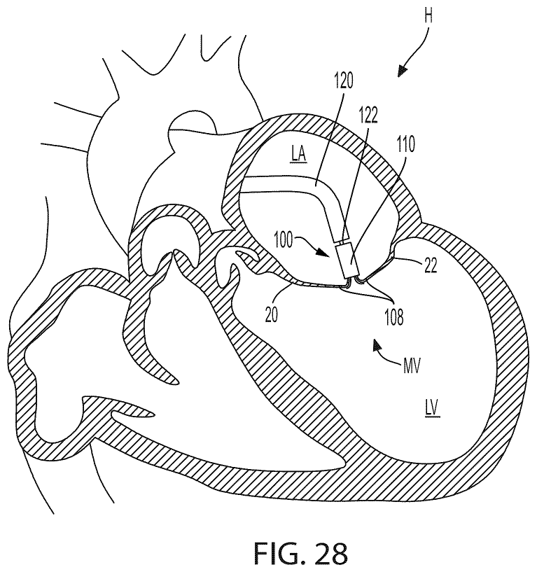

Referring now to FIG. 27, the catheter of the delivery system 120 can be retracted to position the mitral valve leaflets 20, 22 in the anchors 108. Once the leaflets are inside the anchors 108, the actuation element 122 is retracted and/or the catheter of the delivery system 120 is advanced to draw the anchors 108 and leaflets 20, 22 into the capture element 110, capturing a leaflet 20. The actuation element 122 can be further retracted to move the device into the fully closed condition shown in FIG. 28.

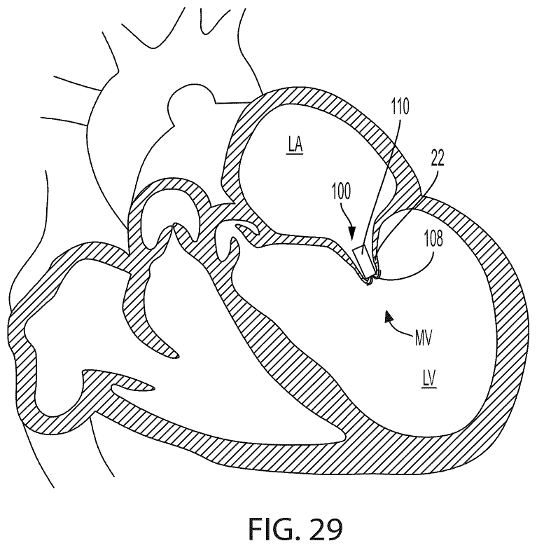

Lastly, as can be seen in FIGS. 28 and 29, the delivery system 120 (e.g., steerable catheter, implant catheter, etc.) and the actuation element 122 are decoupled and retracted and the device or implant 100 is fully closed and deployed in the native mitral valve MV.

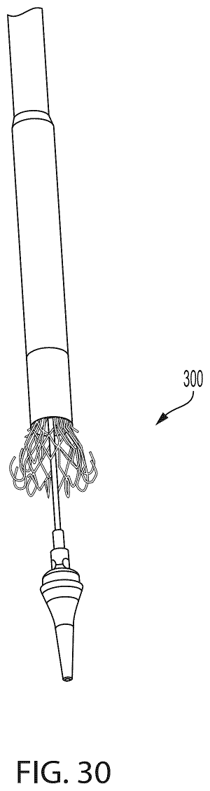

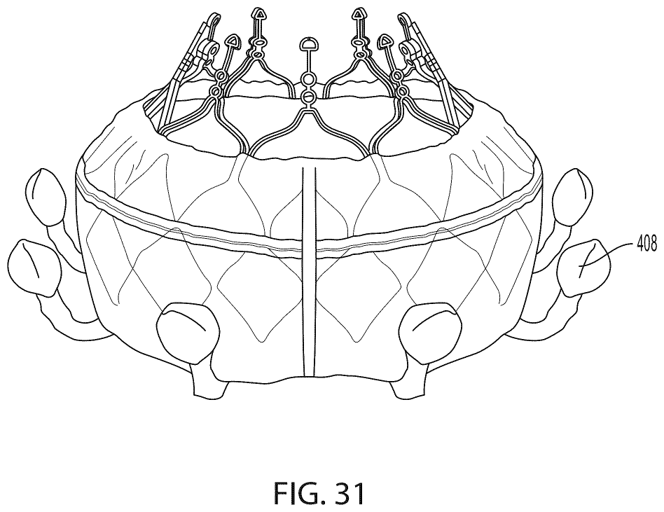

With reference to FIGS. 30-31, the device and components as described herein can take a variety of different forms to be implanted with a variety of different delivery systems. For example, the prosthesis shown in FIG. 31 having anchors 408 can be used in the manner described herein in the system 300 shown in FIG. 30. Additional details and example designs for a prosthesis, such as the prosthesis illustrated by FIG. 31, are described in U.S. Pat. Nos. 8,403,983, 8,414,644, 8,652,203, 10,813,757, and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification.

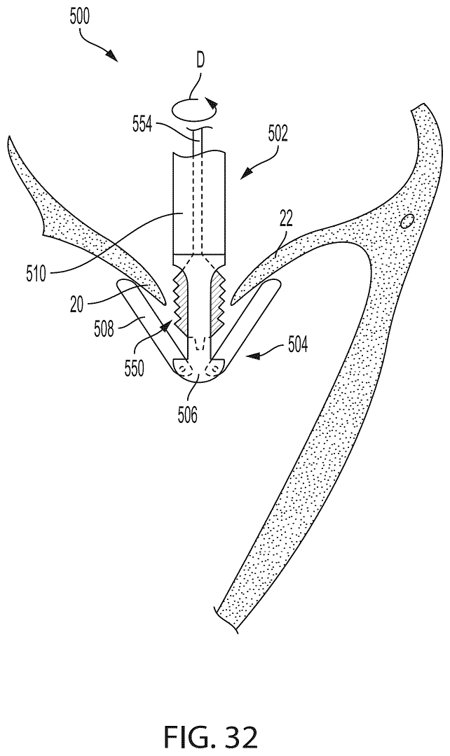

FIGS. 32-34 illustrate examples of valve repair systems 500, 600, and 700 that are configured to draw leaflets 20, 22 into an anchor. The valve repair systems 500, 600, and 700 can take a wide variety of different forms. For example, the systems can have any of the features of any of the systems disclosed herein and/or any of the features of the valve repair systems disclosed by Published PCT Application Nos. WO2018195201, WO2019204559, WO2019051180, WO2018209021, WO2018195215, WO2019139904, WO2020168081, U.S. Patent Publication No. U.S. Pat. No. 20200113676, and U.S. Pat. No. 10,517,726, which are incorporated herein by reference in their entireties. In the examples illustrated by FIGS. 32-34, the valve repair devices include anchors or arms for capturing the leaflets 20, 22 and one or more rotating members that draw or adjust the positions of the native valve leaflets relative to the anchors or arms. The rotating members can take a wide variety of different forms. Any rotational member or apparatus that uses a rotational movement to draw or adjust the positions of the native valve leaflets relative to the anchors or arms can be used. Any of the devices disclosed in Published PCT Application Nos. WO2018195201, WO2019204559, WO2019051180, WO2018209021, WO2018195215, WO2019139904, WO2020168081, U.S. Patent Publication No. U.S. Pat. No. 20200113676, and U.S. Pat. No. 10,517,726 can be modified to include features disclosed herein that draw or adjust the positions of the native valve leaflets relative to the anchors or arms.

With reference to FIG. 32, a system 500 includes a delivery device 502 and an anchor 504. The delivery device 502 can include a catheter 510 and an actuation element 554 or shaft. The illustrated anchor 504 includes a body or base 506 and one or more arms 508. The anchor 504 further includes a rotating member 550 coupled with the actuation element 554 or shaft of the delivery device 502.

The rotating member 550 can take a variety of different forms. The rotating member can be various shapes and sizes. In some implementations, the rotating member can be cylindrical, although the rotating member can also be rectangular, circular, or various other shapes. The rotating member can have a screw-like configuration, with threads, projections, notches, or other gripping members spaced along the rotating member for adjusting the position of the leaflets 20, 22 relative to the arms 508. In the example illustrated by FIG. 32, the rotating member 550 includes an external thread that extends along the surface of the rotating member 550.

The rotating member 550 can be coupled to the actuation element 554 or shaft. The actuation element 554 can take a wide variety of different forms, including a wire, rod, shaft, tube, screw, suture, line, strip, or a combination of these. The actuation element 554 can be connected to the rotating member 550 such that a user can provide a force to the rotating member 550 to cause the rotating member 550 to rotate in a first direction D. When the rotating member 550 rotates in the first direction D, the rotating member 550 makes contact with the leaflets 20, 22 and pulls the leaflets 20, 22 further into the arms 508. The leaflets can then be secured between the rotating member 550 and the arms 508 of the anchor portion 504.

If repositioning of the leaflets relative to the arms 508 and the rotating member 550 is needed (e.g., if the leaflets are pulled to tightly into the anchor portion 604), the rotating member 550 can be rotated in a second direction opposite from the first direction D. When the rotating member 550 is rotated in the second direction, the leaflets are moved out of the arms 508 of the anchor portion 504 until a more desirable fit is achieved.

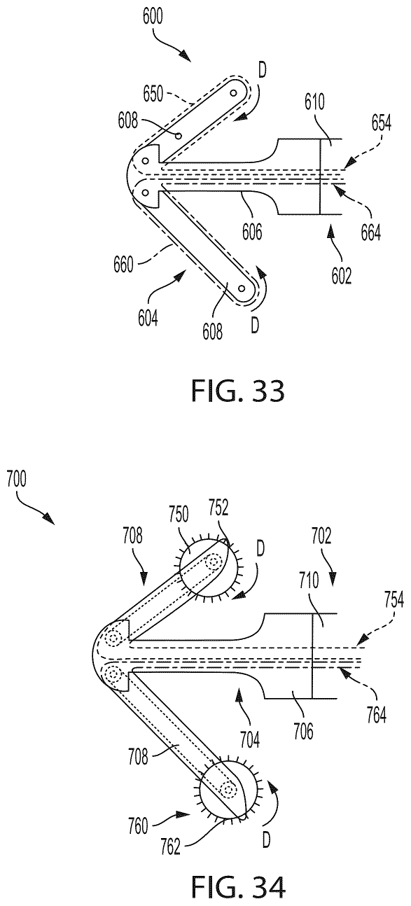

FIG. 33 illustrates an example of a system 600 that includes a delivery device 602 and an anchor 604. The delivery device 602 can include a catheter 610 and two independent actuation elements 654, 664. The illustrated anchor 604 includes a body or base 606 and one or more arms or anchors 608. The anchor 604 further includes first and second rotating/translating members or belt members 650, 660. For example, the anchor portion 604 can include a first rotating/translating member 650 and a second rotating/translating member 660 disposed along a surface of the arms or anchors 608. The rotating members/translating members 650, 660 can include projections, notches, or other friction enhancing elements for coupling with the leaflets.

The rotating/translating members 650, 660 can be coupled to actuation elements 654, 664, respectively, such that a user can provide a force to the rotating/translating members 650, 660. The force applied to the actuation elements 654, 664 can cause the rotating/translating members 650, 660 to move in a first direction D into the arms 608 of the anchor 604. The actuation elements 654, 664 can be independent of one another, such that they can engage simultaneously, at different times, rates and/or amounts. As a result, different lengths or amounts of the first and second leaflets 20, 22 can be drawn into the arms 608 of the anchor 604. Once the leaflets 20, 22 are positioned, the leaflets 20, 22 can be secured between the rotating/translating members 650, 660 and the base 606 of the anchor portion 604.

If repositioning of one or both of the leaflets within the arms 608 of the anchor portion 604 is needed (e.g., if one or more of the leaflets are pulled to tightly into the anchor portion 604), the rotating/translating member(s) 650 and/or 660 can be moved in a second direction opposite from the first direction D. When the rotating/translating member(s) 650 and/or 660 are moved in the second direction, the leaflet(s) are moved out of the arms 608 of the anchor portion 604. The amount of movement of the rotating/translating member(s) 650 and/or 660 is controlled by the actuation elements 654, 664 to control how far the leaflets 20, 22 are released from the arms 608 of the anchor portion 604.

FIG. 34 illustrates an example of a system 700 that is similar to the system 600 of the example of FIG. 34, except the system 700 includes wheels 750, 760 that pull the leaflets into arms 708 of an anchor portion 704. The FIG. 34 system 700 includes a delivery device 702 and an anchor 704. The delivery device 702 can include a catheter 710 and two independent actuation elements 754, 764. The illustrated anchor 704 includes a body or base 706 and one or more arms or anchors 708. The anchor 704 further includes the first and second rotating members or wheels 750, 760. For example, the anchor portion 704 can include a first rotating member or wheel 750 and a second rotating member or wheel 760 disposed at an end of the arms or anchors 708. The rotating members or wheels 750, 760 can include projections, notches, or other friction enhancing elements 752, 762 for engaging with the leaflets 20, 22.

The rotating members or wheels 750, 760 can be coupled to actuation elements 754, 764. The actuation elements can take a wide variety of different forms. For example, the actuation elements 754, 764 can be lines, belts, chains, etc. that can be used to convert a linear motion applied to the actuation elements 754, 764 to rotation of the rotating members or wheels 750, 760. The force applied to the actuation elements 754, 764 can cause the rotating members or wheels 750, 760 to rotate in a first direction D to pull leaflets 20, 22 into the arms 708 of the anchor 704. The actuation elements 754, 764 can be independent of one another, such that the rotating members or wheels 750, 760 can engage leaflet tissue simultaneously, at different times, rates and/or amounts. As a result, different lengths or amounts of the first and second leaflets 20,22 can be drawn into the arms 708 of the anchor 704. Once the leaflets 20, 22 are positioned, the leaflets 20,22 can be secured between the rotating members 750, 760 and the base 706 of the anchor portion 704.

If repositioning of one or both of the leaflets within the arms 708 of the anchor portion 704 is needed (e.g., if the leaflets are pulled to tightly into the anchor portion 704), the rotating member(s) or wheel(s) 750 and/or 760 can be moved in a second direction opposite from the first direction D. When the rotating member(s) or wheel(s) 750 and/or 760 are rotated in the second direction, the leaflet(s) are moved out of the arms 708 of the anchor portion 604. The amount of movement of the rotating member(s) or wheel(s) 750 and/or 760 is controlled by the actuation elements 754, 764 to control how far the leaflet(s) 20, 22 are released from the arms 708 of the anchor portion 704.

Additional information regarding delivery methods for valve repair and replacement devices can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

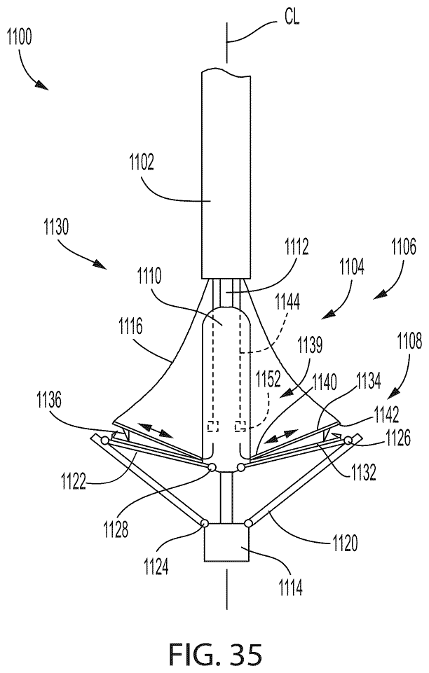

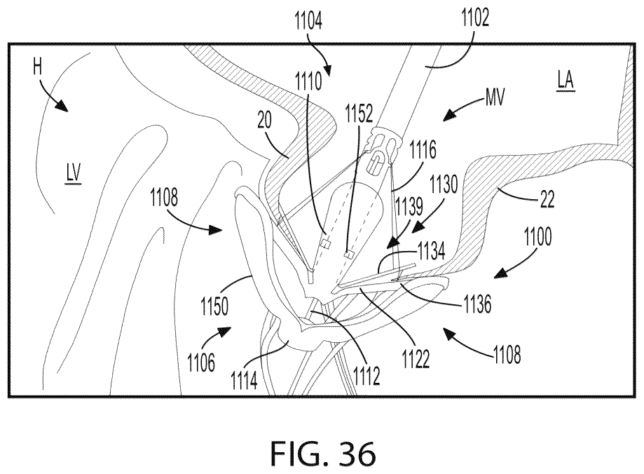

Referring now to FIG. 35, a schematically illustrated implantable device or implant 1100 (e.g., an implantable prosthetic device, a prosthetic spacer device, a valve repair device, etc.) is shown. The implantable device or implant 1100 and other similar devices/implants are described in more detail in PCT patent application publication Nos. WO2018/195215, WO2020/076898, and WO 2019/139904, which are incorporated herein by reference in their entirety. The device 1100 can include any other features for any other implantable device or implant discussed in the present application or the applications cited above, and the device 1100 can be positioned to engage valve tissue (e.g., leaflets 20, 22, 30, 32, 34) as part of any suitable valve repair system (e.g., any valve repair system disclosed in the present application, or the applications cited above).

The device or implant 1100 is deployed from a delivery system or other means for delivery 1102. The delivery system 1102 can comprise one or more of a catheter, a sheath, a guide catheter/sheath, a delivery catheter/sheath, a steerable catheter, an implant catheter, a tube, a channel, a pathway, combinations of these, etc. The device or implant 1100 can include an optional coaptation portion 1104 and an anchor portion 1106.

In some implementations, the coaptation portion 1104 of the device or implant 1100 includes a coaptation element 1110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 1112 (e.g., actuation wire, shaft, tube, hypotube, line, suture, braid, etc.).

In some implementations, the anchor portion 1106 includes one or more anchors 1108 that are actuatable between open and closed conditions and can take a wide variety of forms, such as, for example, paddles, gripping elements, or the like. Actuation of the means for actuating or actuation element 1112 opens and closes the anchor portion 1106 of the device 1100 to grasp the native valve leaflets during implantation.

The means for actuating or actuation element 1112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 1106 relative to the coaptation portion 1104. Or, the actuation element can be unthreaded, such that pushing and/or pulling the actuation element 1112 moves the anchor portion 1106 relative to the coaptation portion 1104.

The anchor portion 1106 and/or anchors can take a variety of different forms. For example, the anchor portion 1106 and/or anchors can take the form of any of the anchors or components of anchors disclosed in the present application or any anchor of a known mitral valve or tricuspid valve repair device. Further, the anchor portion 1106 and/or anchors can take any form that allows the device 1100 to be attached to the leaflets of a mitral valve or a tricuspid valve.

In some implementations, the anchor portion 1106 and/or anchors of the device 1100 include outer paddles 1120 and inner paddles 1122 that are, in some implementations, connected between a cap 1114 and the coaptation element 1110 by portions 1124, 1126, 1128. In some implementations, the device 1100 does not include both outer paddles 1120 and inner paddles 1122. For example, each inner paddle 1120 and outer paddle 1122 combination can be replaced with a single paddle that can be opened and closed. The portions 1124, 1126, 1128 can be jointed and/or flexible to move between various positions. The interconnection of the outer paddles 1120, the inner paddles 1122, the coaptation element 1110, and the cap 1114 by the portions 1124, 1126, and 1128 can constrain the device to the various positions and movements needed to deliver the device to the native valve, open the device, and close the device to secure the device to the leaflets of the native valve.

In some implementations, the delivery system 1102 includes a steerable catheter, implant catheter, and means for actuating or actuation element 1112 (e.g., actuation wire, actuation shaft, actuation tube, actuation rod, etc.). These can be configured to extend through a guide catheter/sheath (e.g., a transseptal sheath, etc.). In some implementations, the means for actuating or actuation element 1112 extends through a delivery catheter and the coaptation element 1110 to the distal end (e.g., a cap 1114 or other attachment portion at the distal connection of the anchor portion 1106). In some implementations, extending and retracting the actuation element 1112 increases and decreases the spacing between the coaptation element 1110 and the distal end of the device (e.g., the cap 1114 or other attachment portion), respectively.

In some implementations, a collar or other attachment element (e.g., clamp, clip, lock, sutures, friction fit, buckle, snap fit, lasso, etc.) removably attaches the coaptation element 1110 to the delivery system 1102, either directly or indirectly, so that the means for actuating or actuation element 1112 slides through the collar or other attachment element and, in some implementations, through a coaptation element 1110 during actuation to open and close the paddles 1120, 1122 of the anchor portion 1106 and/or anchors 1108.

In some implementations, the anchor portion 1106 and/or anchors 1108 can include attachment portions or gripping members 1130 (e.g., arm, clamp, clasp, hook, etc.). The illustrated gripping members can comprise an optional fixed arm 1132, a movable arm 1134, and optional securing elements, friction-enhancing elements, or other means for securing 1136 (e.g., barbs, protrusions, ridges, grooves, textured surfaces, adhesive, etc.). In some implementations, the device 1100 includes a pair of gripping members 1130, each having an optional base or fixed arm 1132, a movable arm 1134, and one or more optional barbs 1136. In some implementations, the fixed arm 1132 is omitted and the movable arm 1134 and the inner paddle 1122 are configured to capture and secure the leaflet of the native valve.

The movable arms 1134 are configured to move between an open position in which the movable arms 1134 extend along the coaptation element 1110 and a closed position, as shown in FIG. 35, in which the movable arms 1134 extend along the fixed arms 1132 (when included) and the inner paddles 1122. In some implementations, the movable arms 1134 are configured to articulate, flex, or pivot to move between the open and the closed position. In some implementations, the movable arms 1134 articulate, flex, or pivot at a position adjacent the distal end of the coaptation elements or adjacent the portion 1128, as shown in FIG. 35. The optional fixed arms 1132 are attached to the inner paddles 1122 and remain stationary or substantially stationary relative to the inner paddles 1122 when the movable arms 1134 are opened to open the gripping members 1130 and expose the optional barb(s) 1136.

In some implementations, the movable arms 1134 can be biased to the closed position. The movable arms 1134 can be biased to the closed position in a variety of ways. For example, the movable arms 1134 can be formed of a shape-memory alloy, such as Nitinol, which is shape set to the closed position or the movable arms 1134 can be biased to the closed position through the use of spring materials, such as steel, other metals, plastics, composites, etc. In some implementations, the gripping members 1130 are opened by applying tension to actuation lines 1116 attached to the movable arms 1134, thereby causing the movable arms 1134 to articulate, flex, or pivot. The actuation lines 1116 extend through the delivery system 1102 (e.g., through a steerable catheter and/or an implant catheter). Other actuation mechanisms are also possible.

The actuation line 1116 can take a wide variety of forms, such as, for example, a line, a suture, a wire, a rod, a catheter, or the like. The gripping members 1130 can be biased so that in the closed position the gripping members 1130 continue to provide a pinching force on the grasped native leaflet. The optional barbs 1136 of the gripping members 1130 can grab, pinch, and/or pierce the native leaflets to further secure the native leaflets.

During implantation, the paddles 1120, 1122 can be opened and closed, for example, to grasp the native leaflets (e.g., native mitral valve leaflets, etc.) between the paddles 1120, 1122 and/or between the paddles 1120, 1122 and a coaptation element 1110 (e.g., a spacer, plug, membrane, gap filler, etc.). The gripping members 1130 can be used to grasp and/or further secure the native leaflets by engaging the leaflets with optional barbs 1136 and pinching the leaflets between the movable and fixed arms 1134, 1132 and/or between the movable arms 1134 and the inner paddle 1122. The optional securing elements 1136 or means for securing (e.g., barbs, friction-enhancing elements, protrusions, ridges, grooves, textured surfaces, adhesive, etc.) of the gripping members 1130 increase friction with the leaflets or can partially and/or completely puncture the leaflets.

In some implementations, the actuation lines 1116 can be actuated separately so that each gripping member 1130 can be opened and closed separately. Separate operation allows one leaflet to be grasped at a time, or for the repositioning of a gripping member 1130 on a leaflet that was insufficiently grasped, without altering a successful grasp on the other leaflet. In some implementations, the actuation lines 1116 can be actuated simultaneously so that the gripping member 1130 can be opened and closed together (e.g., to simultaneously capture two or more leaflets).

In some implementations, the gripping members 1130 can be opened and closed relative to the position of the inner paddle 1122 (as long as the inner paddle is in an open or at least partially open position), thereby allowing leaflets to be grasped in a variety of positions as the particular situation requires.

In some implementations, the device 1100 includes a leaflet repositioning device 1139 configured to reposition a captured leaflet 20, 22 relative to the device 1100. Repositioning a captured leaflet 20, 22 can allow the device 1100 to reposition a leaflet that was insufficiently grasped to an improved captured position without having to release and reposition the gripping member 1130.

The leaflet repositioning device can be configured in a variety of ways. In some implementations, the leaflet repositioning device 1139 allows each of the movable arms 1134 to be separately retracted (i.e., move inward toward a centerline CL of the device 1100) while in the closed position. Thus, the effective length (e.g., the exposed length) of the movable arm 1134 can be customized in order to reposition the captured leaflet relative to the optional fixed arm 1132 and/or the inner paddle 1122.

As shown in FIG. 35, each movable arm 1134 has a proximal end 1140 and a distal end 1142. Each of the movable arms 1134 can be separately retracted while in the closed position such that the distal end 1142 of the movable arm 1134 moves inward toward the coaptation element 1110 or a centerline CL of the device. The implantable device 1100 can be configured in a variety of way to allow for retraction of the movable arms 1134. In some implementations, the proximal end 1140 of each movable arm 1134 is connected to a corresponding retraction element 1144. The retraction element 1144 can take a wide variety of forms, such as, for example, a line, a suture, a wire, a rod, a tube, a catheter, or the like. The retraction element 1144 can connect to the proximal end 1140 of each movable arm 1134 in any suitable manner or with any type of connecting device, lock, fastener, etc.

Retraction of the movable arm 1134 allows a leaflet that was insufficiently grasped to be pulled inward toward the coaptation element 1110 to improve capture of the leaflet. Referring to FIG. 36, the implantable device 1100 is shown positioned at the mitral valve annulus with the moveable arms 1134 in the closed position and with leaflets 20, 22 captured by the gripping members 1130.

In some implementations, the implantable device 1100 can also include an optional cover 1150. In some implementations, the cover 1150 can be disposed on the coaptation element 1110 and/or the outer and inner paddles 1120, 1122. The cover 1150 can be configured to prevent, inhibit, or reduce blood-flow through the device or implant 1100 and/or to promote native tissue ingrowth. In some implementations, the cover 1150 can be a cloth or fabric such as PET, velour, or other suitable fabric. In some implementations, in lieu of or in addition to a fabric, the cover 1150 can include a coating (e.g., polymeric) that is applied to the implantable device or implant 1100.

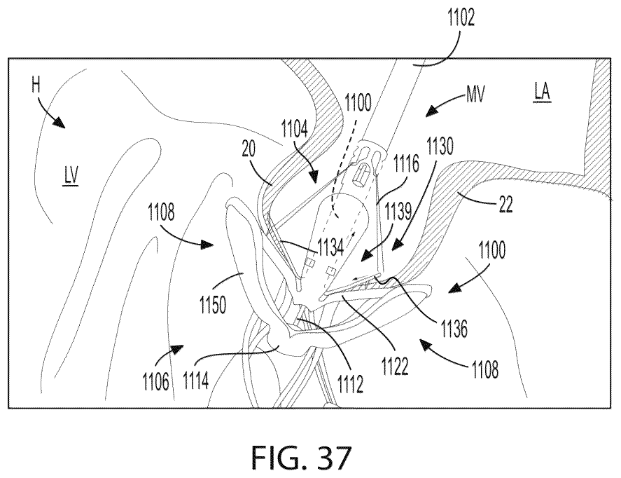

As shown in FIG. 36, one leaflet 20 is captured sufficiently (e.g., sufficient depth within the gripping member 1130) but the other leaflet 22 is insufficiently captured (e.g., insufficient depth within the gripping member 1130). In particular, the insufficiently captured leaflet 22 is only captured at the distal end 1142 of the movable arm 1134. To improve the capture of this leaflet 22 when the paddles 1122 are closed, the moveable arm 1134 can be retracted, as shown in FIG. 37. As shown by arrow A, tension can be applied to the retraction element 1144 to pull the retraction element 1144 proximally such that the proximal end 1140 of the movable arm 1134 is pulled into the optional coaptation element 1110. As a result, the movable arm 1134, while remaining in the closed position, moves inward relative to the fixed arm 1132 and/or paddle 1122 (i.e., moves to a retracted position). Since the leaflet 22 is connected to the movable arm 1134 via the securing element 1136 on the movable arm 1134, when the movable arm 1134 moves inward, the movable arm 1134 pulls the leaflet 22 inward with it.

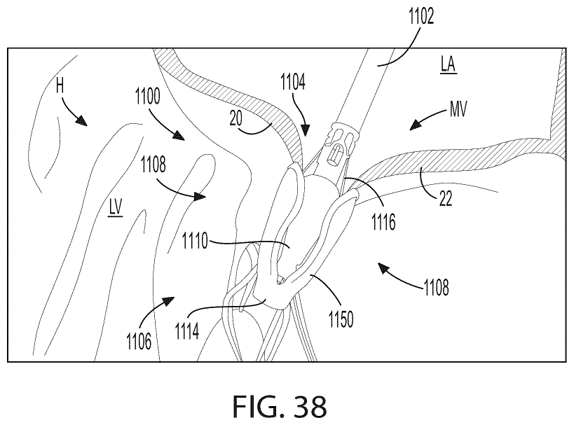

In some implementations, one or more locking elements 1152 can be operatively associated with each of the retraction elements 1144 and/or each of the movable arms 1134. For example, one locking element 1152 can be configured to lock one of the moveable arms 1134 in position after the movable arm 1134 has been retracted (i.e., fixes the effective length of the moveable arm 1134). Likewise, another locking element 1152 can be configured to lock the other of the moveable arms 1134 in position after the other movable arm 1134 has been retracted. Thus, the leaflet 22 can be pulled inward to a sufficient depth by movement of the movable arm 1134 and locked into place relative to the inner paddles 1122 such that when the paddles 1120, 1122 are closed, the leaflets 20, 22 are held securely by the implantable device 1100 as shown by FIG. 38.

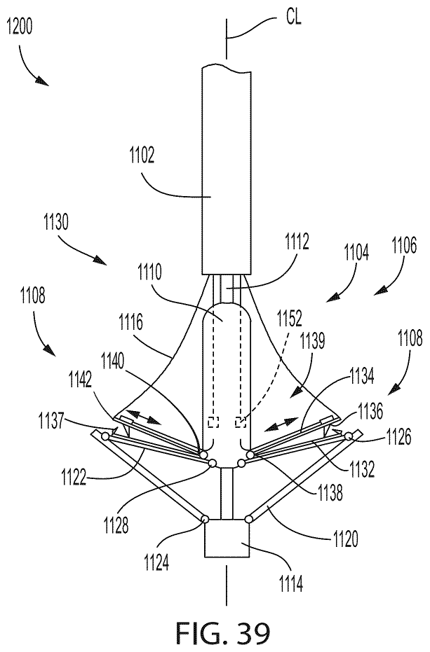

Referring now to FIGS. 39-42, a schematically illustrated example device or implant 1200 (e.g., a repair device, an implantable device, an implantable prosthetic device, a prosthetic spacer device, a valve repair device, etc.) is shown. The device or implant 1200 corresponds to the previously disclosed device or implant illustrated in FIGS. 35-38 and the description of the device or implant 1100 applies equally to the device or implant 1200 with reference numbers of like elements kept the same. The device or implant 1200 can include any feature of any device or implant 1200 discussed in the present application or the applications cited above. The device 1200 can be positioned to engage valve tissue (e.g., leaflets 20, 22, 30, 32, 34) as part of any suitable valve repair system (e.g., any valve repair system disclosed in the present application, or the applications cited above).

In some implementations, the device 1200 includes a coaptation portion 1104 and an anchor portion 1106. The coaptation portion 1104 includes a coaptation element 1110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 1112 (e.g., actuation wire, shaft, tube, hypotube, line, suture, braid, etc.).

In some implementations, the anchor portion 1106 includes one or more anchors 1108 that are actuatable between open and closed conditions and can take a wide variety of forms, such as, for example, paddles, gripping elements, or the like. Actuation of the means for actuating or actuation element 1112 opens and closes the anchor portion 1106 of the device 1100 to grasp the native valve leaflets during implantation. The means for actuating or actuation element 1112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations.

The anchor portion 1106 and/or anchors can take a variety of different forms. For example, the anchor portion 1106 and/or anchors can take the form of any of the anchors or components of anchors disclosed in the present application or any anchor of a known mitral valve or tricuspid valve repair device. Further, the anchor portion 1106 and/or anchors can take any form that allows the device 1100 to be attached to the leaflets of a mitral valve or a tricuspid valve.

In some implementations, the anchor portion 1106 and/or anchors of the device 1200 include outer paddles 1120 and inner paddles 1122 that are, in some implementations, connected between a cap 1114 and the coaptation element 1110 by portions 1124, 1126, 1128. However, in some implementations, the device 1100 does not include both outer paddles 1120 and inner paddles 1122. For example, each inner paddle 1120 and outer paddle 1122 combination can be replaced with a single paddle that can be opened and closed.

In some implementations, the anchor portion 1106 and/or anchors 1108 can include attachment portions or gripping members. The illustrated gripping members can comprise gripping members 1130 that include an optional base or fixed arm 1132, a movable arm 1134, an optional securing element or means for securing 1136 (e.g., barbs, friction-enhancing elements, protrusions, ridges, grooves, textured surfaces, adhesive, etc.), and a joint portion 1138. When included, the optional fixed arms 1132 can be attached to the inner paddles 1122. In some implementations, the fixed arms 1132 are attached to the inner paddles 1122 with the joint portion 1138 disposed proximate an optional coaptation element 1110.

In some implementations, the joint portion 1138 provides a spring force between the fixed and movable arms 1132, 1134 of the gripping member 1130 and/or provides a spring force between the movable arms 1134 and the inner paddles 1120. The joint portion 1138 can be any suitable joint, such as a flexible joint, a spring joint, a pivot joint, or the like. In some implementations, the joint portion 1138 is a flexible piece of material integrally formed with the fixed and movable arms 1132, 1134. The fixed arms 1132 are attached to the inner paddles 1122 and remain stationary or substantially stationary relative to the inner paddles 1122 when the movable arms 1134 are opened to open the gripping members 1130 and expose the optional securing element 1136.

The movable arms 1134 are configured to move between an open position in which the movable arms 1134 extend along the coaptation element 1110 and a closed position, as shown in FIG. 39, in which the movable arms 1134 extend along the optional fixed arms 1132 and/or the inner paddles 1122. The fixed arms 1132 are attached to the inner paddles 1122 and remain stationary or substantially stationary relative to the inner paddles 1122 when the movable arms 1134 are opened to open the gripping members 1130 and expose the optional securing element or means for securing 1136.

In some implementations, the gripping members 1130 are opened by applying tension to actuation lines 1116 attached to the movable arms 1134, thereby causing the movable arms 1134 to articulate, flex, or pivot on the joint portions 1138. The actuation lines 1116 extend through the delivery system 1102 (e.g., through a steerable catheter and/or an implant catheter). Other actuation mechanisms are also possible. The actuation line 1116 can take a wide variety of forms, such as, for example, a line, a suture, a wire, a rod, a catheter, or the like. The gripping members 1130 can be biased so that in the closed position the gripping members 1130 continue to provide a pinching force on the grasped native leaflet. This pinching force remains constant regardless of the position of the inner paddles 1122. The optional securing element or means for securing 1136 of the gripping members 1130 can grab, pinch, and/or pierce the native leaflets to further secure the native leaflets.

In some implementations, during implantation, the paddles 1120, 1122 can be opened and closed, for example, to grasp the native leaflets (e.g., native mitral valve leaflets, etc.) between the paddles 1120, 1122 and/or between the paddles 1120, 1122 and a coaptation element 1110 (e.g., a spacer, plug, membrane, gap filler, etc.). The gripping members 1130 can be used to grasp and/or further secure the native leaflets by engaging the leaflets with optional securing elements 1136 and pinching the leaflets between the movable and fixed arms 1134, 1132. The optional securing elements 1136 (e.g., protrusions, ridges, grooves, textured surfaces, adhesive, etc.) of the gripping members 1130 increase friction with the leaflets or can partially or completely puncture the leaflets.

In some implementations, the actuation lines 1116 can be actuated separately so that each gripping member 1130 can be opened and closed separately. Separate operation allows one leaflet to be grasped at a time, or for the repositioning of a gripping member 1130 on a leaflet that was insufficiently grasped, without altering a successful grasp on the other leaflet. In some implementations, the gripping members 1130 can be opened and closed relative to the position of the inner paddle 1122 (as long as the inner paddle is in an open or at least partially open position), thereby allowing leaflets to be grasped in a variety of positions as the particular situation requires.

In some implementations, the device 1200 includes a leaflet repositioning device 1139 configured to reposition a captured leaflet 20, 22 relative to the device 1200. Repositioning a captured leaflet 20, 22 can allow the device 1200 to reposition a leaflet that was insufficiently grasped to a more secure captured position without having to release and reposition the gripping member 1130. The leaflet repositioning device can be configured in a variety of ways. In some implementations, the leaflet repositioning device 1139 allows the movable arm 1134 and attached securing element 1136 to be drawn inward (i.e., toward a centerline CL of the device 1200) and/or outward (i.e., away from the centerline CL of the device 1200) in order to reposition the captured leaflet relative to the fixed arm 1132 and/or the inner paddle 1122. In some implementations, the leaflet repositioning device 1139 allows the securing element 1136 to be moved inward (i.e., toward a centerline CL of the device 1200) and outward (i.e., away from the centerline CL of the device 1200) along the movable arm 1134 in order to reposition the captured leaflet relative to the fixed arm 1132 and/or the inner paddle 1122.

As shown in FIG. 39, each movable arm 1134 has a proximal end 1140 and a distal end 1142. In some implementations, each of the movable arms 1134 and attached securing elements 1136 can be separately drawn inward and released outward while in the closed position. In some implementations, the securing element 1136 on each of the movable arms 1134 can be separately moved along the movable arm 1134 toward and away from the proximal end 1140 while in the closed position. For example, the securing element 1136 on each of the movable arms 1134 can be moved back and forth between a distal position on the movable arm 1134 to a proximal position that is closer to the proximal end 1140 than the distal position is.

The implantable device 1200 can be configured in a variety of ways to allow for movement of the securing element 1136 along the movable arm 1134. In some implementations, the securing element 1136 of each movable arm 1134 is connected to a corresponding retraction element 1144. The retraction element 1144 can take a wide variety of forms, such as, for example, a line, a suture, a wire, a rod, a catheter, or the like. The retraction element 1144 can connect to the securing element 1136 of each movable arm 1134 in any suitable manner or by any connecting device. For example, the securing element can be disposed on or comprise a ring, collar, etc. that is slidably disposed on the movable arm 1134.

Inward movement of the securing element 1136 toward the centerline CL of the device 1200 allows a leaflet that was insufficiently grasped to be pulled inward toward the coaptation element 1110 or the centerline CL of the device 1200 to improve capture of the leaflet. In some implementations, one or more locking elements 1152 can be operatively associated with each of the retraction elements 1144, each of the movable arms 1134, and/or each securing element 1136. For example, the one or more locking elements 1152 can be configured to lock the securing element 1136 in position after the securing element 1136 has been moved inward. Thus, the insufficiently grasped leaflet can be pulled inward to a sufficient depth by movement of the securing element 1136 and locked into place relative to the inner paddles 1122 such that when the paddles 1120, 1122 are closed, the leaflets 20, 22 are held securely by the implantable device 1200.

In some implementations, the leaflet repositioning device 1139 is configured as a ratcheting mechanism, or similar device or mechanism. That is, after the securing element 1136 is pulled inward toward the centerline CL of the device 1200, the securing element 1136 on the movable arm 1134 can, if desired, be released from the leaflet and return to the distal position of on the movable arm 1134. The securing element 1136 can then be reengaged with the leaflet and pulled inward toward the centerline CL of the device 1200 to the proximal position again. In this manner, the leaflet can be incrementally pulled inward toward the centerline CL of the device 1200 until considered to be sufficiently grasped and the overall length of travel of the securing element 1136 can be short since the inward movement can be repeated.

In some implementations, the leaflet repositioning device 1139 can be configured as a ratcheting mechanism by including a second securing element 1137 (e.g., protrusions, ridges, grooves, textured surfaces, adhesive, etc.). That is, after the securing element 1136 is pulled inward toward the centerline CL of the device 1200, the second securing element 1137 holds the position of the valve leaflet 20, 22. Then, the securing element 1136 can slide over the leaflet and be moved back to the distal position. The securing element 1136 can then be reengaged with the leaflet and pull the leaflet inward toward the centerline CL over the second securing element 1137 to the proximal position again and the second securing element 1137 can hold the position of the valve leaflet again. In this manner, the leaflet can be incrementally pulled inward toward the centerline CL of the device 1200 until considered to be sufficiently grasped. In some implementations, the overall length of travel of the securing element 1136 can be short since the inward movement can be repeated.

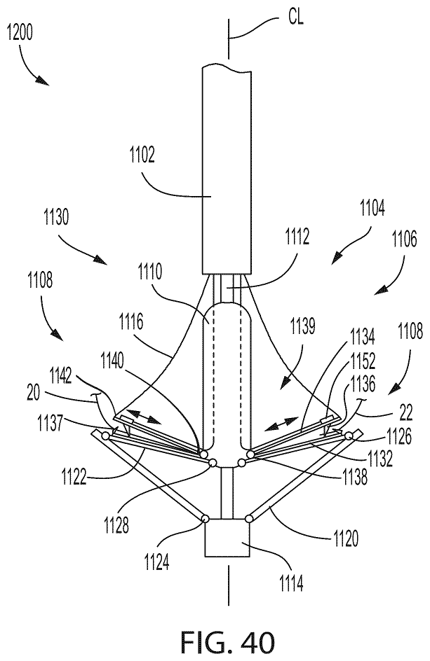

Referring to FIG. 40, the implantable device 1200 is shown positioned at the mitral valve annulus with the moveable arms 1134 in the closed position and with leaflets 20, 22 captured by the gripping members 1130. In the illustrated implementation, one leaflet 20 is captured sufficiently (e.g., sufficient depth within the gripping member 1130) but the other leaflet 22 is insufficiently captured (e.g., insufficient depth within the gripping member 1130). In particular, the insufficiently clasped leaflet 22 is only captured at the distal end 1142 of the movable arm 1134. In some implementations, such as the illustrated implementation, both the movable arms 1134 and the fixed arms 1132 have securing elements 1136, 1137 to engage the leaflets 20, 22.

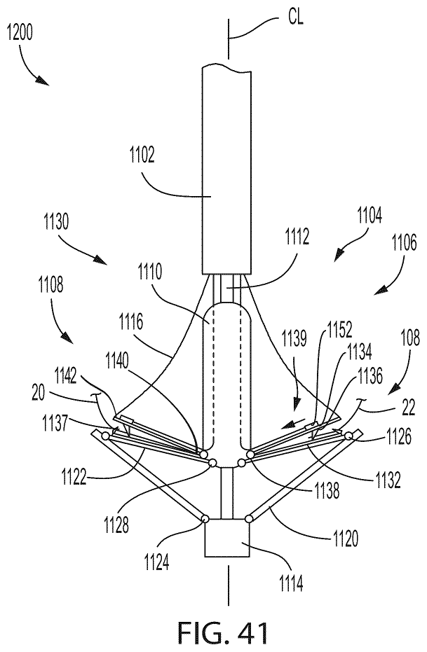

To improve the capture of this leaflet 22, the securing element 1136 on the movable arm 1134 can be drawn inward, as shown in FIG. 41, from the distal position (as shown in FIG. 40) to a proximal position. As shown by the arrows in FIG. 41, tension can be applied to the retraction element 1144 to pull the retraction element 1144 proximally such that the securing element 1136 on the movable arm 1134 is pulled inward, relative to the movable arm 1134, toward the centerline CL of the device 1200. Since the leaflet 22 is connected to the movable arm 1134 via the securing element 1136 on the movable arm 1134, the leaflet 22 is drawn inward by the securing element 1136.

The securing element 1137 on the fixed arm 1132 can be configured to minimally resist, or not resist, movement of the leaflet 22 in the inward direction so as not to damage the leaflet 22 or impede movement of the leaflet inward. For example, the fixed arm 1132 can include barbs that are angled inward to allow the leaflet 22 to slide over the barbs when moving in an inward direction.

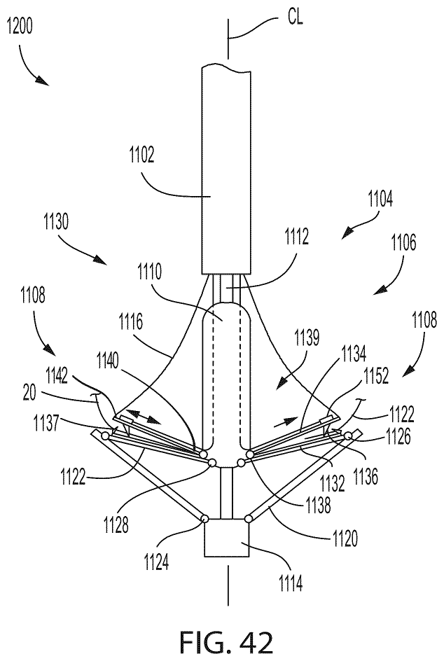

Referring to FIG. 42, after the leaflet 22 has been drawn inward, the securing element 1136 on the movable arm 1134 can release the leaflet 22 and move outward, relative to the movable arm 1134, back to the distal position while the securing element 1137 holds the position of the leaflet 22. For example, the movable arm 1134 can include barbs that are angled inward to allow the leaflet 22 to slide over the barbs when the barbs move outward. The securing element 1136 on the movable arm 1134 can be moved outward in a variety of ways. For example, the securing element 1136 can be biased outward to the distal position using spring materials or pushed outward by the retraction element 1144 or another pusher.

When the securing element 1136 on the movable arm 1134 moves outward to the distal position, the leaflet 22 is held in place by the securing element 1137 on the fixed arm 1132. From the distal position, the securing element 1136 on the movable arm 1134 can reengage the leaflet 22 be pulled inward again to the proximal position to further draw the leaflet inward. This ratcheting action can be configured to allow the securing element 1136 on the movable arm 1134 to repeatedly be moved between the distal position and the proximal position to incrementally draw the leaflet inward until the leaflet is sufficiently grasped and locked into place relative to the inner paddles 1122 such that when the paddles 1120, 1122 are closed, the leaflets 20, 22 are held securely by the implantable device 1200.

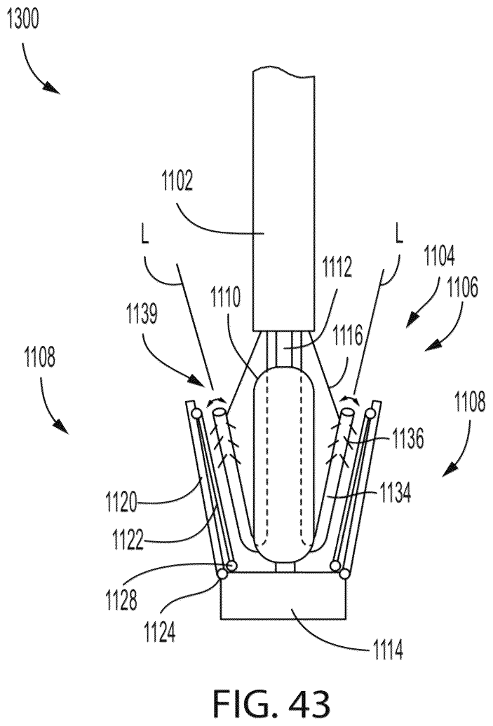

Referring to FIGS. 43-45, a schematically illustrated device or implant 1300 (e.g., a repair device, an implantable device, an implantable prosthetic device, a prosthetic spacer device, a valve repair device, etc.) is shown. The device or implant 1300 corresponds to the previously disclosed device or implant illustrated in FIGS. 39-42 and the description of the device or implant 1100 applies equally to the device or implant 1300 with reference numbers of like elements kept the same. The device or implant 1300 can include any feature of any device or implant 1100 discussed in the present application or the applications cited above. The device 1300 can be positioned to engage valve tissue (e.g., leaflets 20, 22, 30, 32, 34) as part of any suitable valve repair system (e.g., any valve repair system disclosed in the present application, or the applications cited above).

In some implementations, the device 1300 includes a coaptation portion 1104 and an optional anchor portion 1106. In some implementations, the coaptation portion 1104 includes a coaptation element 1110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 1112 (e.g., actuation wire, shaft, tube, hypotube, line, suture, braid, etc.). The anchor portion 1106 includes one or more anchors 1108 that are actuatable between open and closed conditions and can take a wide variety of forms, such as, for example, paddles, gripping elements, or the like.

In some implementations, actuation of the means for actuating or actuation element 1112 opens and closes the anchor portion 1106 of the device 1100 to grasp the native valve leaflets during implantation. The means for actuating or actuation element 1112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations.

The anchor portion 1106 and/or anchors can take a variety of different forms. For example, the anchor portion 1106 and/or anchors can take the form of any of the anchors or components of anchors disclosed in the present application or any anchor of a known mitral valve or tricuspid valve repair device. Further, the anchor portion 1106 and/or anchors can take any form that allows the device 1100 to be attached to the leaflets of a mitral valve or a tricuspid valve.

In some implementations, the anchor portion 1106 and/or anchors of the device 1300 include outer paddles 1120 and inner paddles 1122 that are, in some implementations, connected between a cap 1114 and the coaptation element 1110 by portions 1124, 1126, 1128. However, in some implementations, the device 1100 does not include both outer paddles 1120 and inner paddles 1122. For example, each inner paddle 1120 and outer paddle 1122 combination can be replaced with a single paddle that can be opened and closed. In some implementations, the anchor portion 1106 and/or anchors 1108 can include attachment portions or gripping members. In some implementations, as illustrated, the gripping members 1130 include a movable arm 1134 and/or optional securing element 1136 (e.g., barbs, friction-enhancing elements, protrusions, ridges, grooves, textured surfaces, adhesive, etc.).

The movable arms 1134 are configured to move between an open position in which the movable arms 1134 extend along the coaptation element 1110 and a closed position, as shown in FIG. 39, in which the movable arms 1134 extend along the inner paddles 1122. The movable arms 1134 are opened to open the gripping members 1130 and expose the optional securing element 1136. In some implementations, the movable arms 1134 can be biased to the closed position. The movable arms 1134 can be biased to the closed position in a variety of ways. For example, the movable arms 1134 can be formed of a shape-memory alloy, such as Nitinol, which is shape set to the closed position or the movable arms 1134 can be biased to the closed position through the use of spring materials, such as steel, other metals, plastics, composites, etc.

In some implementations, the gripping members 1130 are opened by applying tension to actuation lines 1116 attached to the movable arms 1134, thereby causing the movable arms 1134 to articulate, flex, or pivot. The actuation lines 1116 extend through the delivery system 1102 (e.g., through a steerable catheter and/or an implant catheter). Other actuation mechanisms are also possible. The actuation line 1116 can take a wide variety of forms, such as, for example, a line, a suture, a wire, a rod, a catheter, or the like. The gripping members 1130 can be biased so that in the closed position the gripping members 1130 continue to provide a pinching force on the grasped native leaflet. This pinching force remains constant regardless of the position of the inner paddles 1122. The optional securing element 1136 of the gripping members 1130 can grab, pinch, and/or pierce the native leaflets to further secure the native leaflets.

In some implementations, during implantation, the paddles 1120, 1122 can be opened and closed, for example, to grasp the native leaflets (e.g., native mitral valve leaflets, etc.) between the paddles 1120, 1122 and/or between the paddles 1120, 1122 and a coaptation element 1110 (e.g., a spacer, plug, membrane, gap filler, etc.). In some implementations, the gripping members 1130 can be used to grasp and/or further secure the native leaflets by engaging the leaflets with optional securing element 1136. The optional securing element of the gripping members 1130 increase friction with the leaflets or can partially or completely puncture the leaflets.

In some implementations, the actuation lines 1116 can be actuated separately so that each gripping member 1130 can be opened and closed separately. Separate operation allows one leaflet to be grasped at a time, or for the repositioning of a gripping member 1130 on a leaflet that was insufficiently grasped, without altering a successful grasp on the other leaflet. The gripping members 1130 can be opened and closed relative to the position of the inner paddle 1122 (as long as the inner paddle is in an open or at least partially open position), thereby allowing leaflets to be grasped in a variety of positions as the particular situation requires.

In some implementations, the device 1300 includes a leaflet repositioning device 1139 configured to reposition the device 1300 on one or more of the captured leaflets 20, 22. Repositioning the device 1300 on a leaflet 20, 22 can allow the device 1300 to be repositioned relative to one or both grasped leaflets 20, 22 by translating the device 1300 along one or more of the leaflets without releasing the device 1300. The leaflet repositioning device 1139 can be configured in a variety of ways. In some implementations, the leaflet repositioning device 1139 can include one or more movable arms 1134 that are rotatable about a longitudinal axis L of the movable arm 1134. In some implementations, the leaflet repositioning device 1139 can be configured to rotate one of the movable arms 1134. In some implementations, the leaflet repositioning device 1139 can be configured to rotate both of the movable arms 1134, either separately or in unison, such as with a geared system. In some implementations, the leaflet repositioning device 1139 is configured to rotate one movable arm 1134 clockwise and the other movable arm 1134 counterclockwise. In some implementations, the movable arms 1134 are shaped as cylindrical rods or tubes with the optional securing element 1136 disposed circumferentially around an exterior surface of the movable arms 1134.

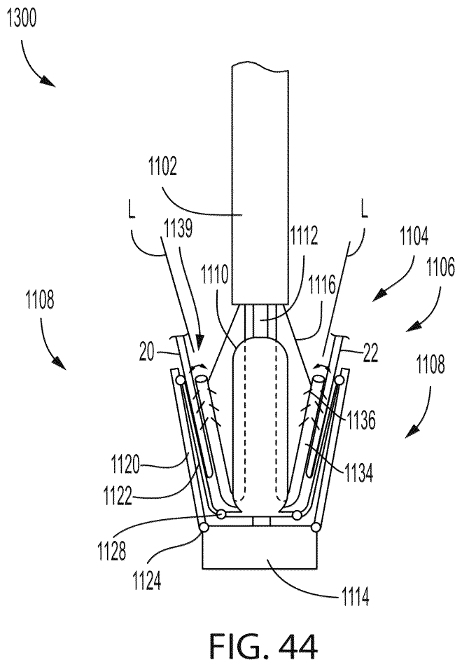

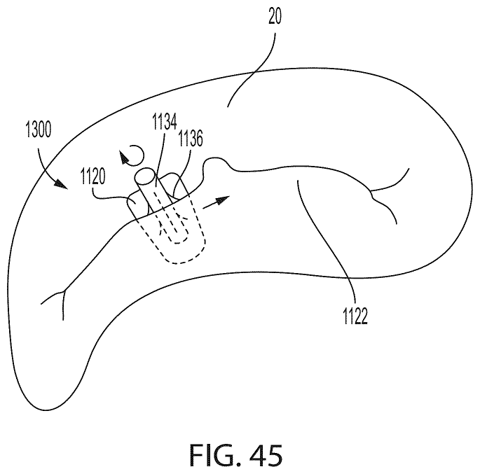

As shown in FIGS. 44-45, with the device 1300 closed and the leaflets 20, 22 captured between the movable arms 1134 and the inner paddles 1122, one or both of the movable arms 1134 can be rotated. By rotating both of the movable arms 1134, one clockwise and the other counterclockwise at the same rotational speed, the device 1300 can translate laterally along the leaflets 20, 22 (e.g., along the commissure of the leaflets), as shown in FIG. 45 (only a single rotatable movable arm 1134 is illustrated for simplicity). In this way, if mitral regurgitation is noticed laterally of where the device 1300 is deployed, the device 1300 can be moved laterally to the location of the mitral regurgitation to address the problem.

In some implementations, one or more locking elements (e.g., clamp, lock, catch, setscrew, etc.) can be operatively associated with each of the movable arms 1134. For example, one locking element (not shown) can be configured to lock one of the moveable arms 1134 in a rotational position after the movable arm 1134 has been rotated. Likewise, another locking element (not shown) can be configured to lock the other of the moveable arms 1134 in a rotational position after the other movable arm 1134 has been rotated. Thus, the leaflets 20, 22 can be repositioned relative to the device 1300 and locked into place relative to the inner paddles 1122 such that when the paddles 1120, 1122 are closed, the leaflets 20, 22 are held securely by the implantable device 1100.

Any of the various systems, devices, apparatuses, components, etc. in this disclosure (including any of the examples below) can be sterilized (e.g., with heat, radiation, ethylene oxide, hydrogen peroxide, etc.) to ensure they are safe for use with patients, and the methods herein can comprise sterilization of the associated system, device, apparatus, components, etc. (e.g., with heat, radiation, ethylene oxide, hydrogen peroxide, etc.).

Examples (some non-limiting examples of the concepts herein are recited below):

Example 1. An implantable device comprising: (i) a capture element having a first end, a second end and a cavity between the first end and the second end; (ii) an anchor portion comprising one or more anchors, wherein the anchor portion is disposed at least partially in the capture element, wherein the anchors are configured to capture one or more leaflets of a native heart valve, wherein the anchor portion is extendable out of the cavity of the capture element and retractable into the cavity of the capture element, and wherein the capture element and the anchor portion are configured to draw native valve leaflet tissue into the cavity when the anchor portion is retracted into the cavity of the capture element.

Example 2. The implantable device of Example 1, wherein the capture element is cylindrical in shape.

Example 3. The implantable device of any one of Examples 1-2, wherein the capture element is round in cross section.

Example 4. The implantable device of any one of Examples 1-3, wherein the inner cavity extends from the first end of the capture element to the second end of the capture element.]

Example 5. The implantable device of Example 4, wherein in the closed position, the anchor portion is housed entirely within the inner cavity.

Example 6. The implantable device of Example 4, wherein in the open position, the anchor portion is housed at least partially outside of the inner cavity.

Example 7. The implantable device of Example 4, wherein the capture element is impervious to blood

Example 8. The implantable device of any one of Examples 1-7, wherein the capture element comprises an opening in an end wall at the first end of the capture element.

Example 9. The implantable device of any one of Examples 1-8, wherein the second end of the capture element is open, such that the anchor portion can be moved in and out of the capture element from the second end.

Example 10. The implantable device of Example 4, wherein the capture element comprises a one-way valve.

Example 11. The implantable device of any one of Examples 1-10, wherein the anchors are made from a flexible or expandable material.

Example 12. The implantable device of any one of Examples 1-11, wherein the anchor portion comprises a body coupled to the anchors.

Example 13. The implantable device of any one of Examples 1-12, wherein the anchor portion is expandable.

Example 14. The implantable device of any one of Examples 1-13, wherein the capture element is removably attached to a delivery catheter.

Example 15. The implantable device of Example 14, wherein the anchor portion is removably attached to an actuation element.

Example 16. The implantable device of Example 15, wherein the actuation element is disposed radially inward of the delivery catheter.

Example 17. The implantable device of Example 15, wherein the actuation element is attached to a collar of the anchor portion.

Example 18. The implantable device of Example 15, wherein movement of the actuation element can move the anchor portion between a closed position and an open position.

Example 19. The implantable device of Example 15, wherein the actuation element is connected to the anchor portion such that a user can provide a tensioning force to the actuation element to cause the anchor portion to move from an expanded position having an expanded width to a narrowed position having a narrowed width, wherein the expanded width is greater than the narrowed width.

Example 20. An implantable device comprising: (i) a capture element having a first end, a second end, and a cavity between the first end and the second end, and (ii) an anchor portion comprising an inner anchor body and an outer anchor body, wherein the anchor portion is disposed at least partially in the capture element, wherein the outer anchor body is extendable out of the cavity of the capture element and retractable into the cavity of the capture element, wherein the inner anchor body is extendable out of the outer anchor body and retractable into the outer anchor body, wherein the inner anchor body and outer anchor body are configured to capture native valve leaflet tissue therebetween, and wherein the capture element and the anchor portion are configured to draw native valve leaflet tissue into the cavity when the anchor portion is retracted into the cavity of the capture element.

Example 21. The implantable device of Example 20, wherein the capture element is cylindrical in shape.

Example 22. The implantable device of any one of Examples 20-21, wherein the capture element is round in cross section.

Example 23. The implantable device of any one of Examples 20-22, wherein the inner cavity extends from the first end of the capture element to the second end of the capture element.

Example 24. The implantable device of Example 23, wherein in the closed position, the anchor portion is housed entirely within the inner cavity.

Example 25. The implantable device of Example 23, wherein in the open position, the anchor portion is housed at least partially outside of the inner cavity.

Example 26. The implantable device of Example 23, wherein the capture element is impervious to blood.

Example 27. The implantable device of any one of Examples 20-26, wherein the capture element comprises an opening in the first end of the capture element.

Example 28. The implantable device of any one of Examples 20-27, wherein the second end of the capture element is open, such that the anchor portion can be moved in and out of the capture element from the second end.

Example 29. The implantable device of Example 23, wherein the capture element comprises a one-way valve.

Example 30. The implantable device of any one of Examples 20-29, wherein the anchors are made from a flexible or expandable material.

Example 31. The implantable device of any one of Examples 20-30, wherein the anchor portion is expandable.

Example 32. The implantable device of any one of Examples 20-31, wherein the capture element is removably attached to a delivery catheter.

Example 33. The implantable device of Example 32, wherein the anchor portion is removably attached to one or more actuation element.

Example 34. The implantable device of Example 32, wherein the inner anchor body is removably attached to an inner actuation element.

Example 35. The implantable device of Example 34, wherein the outer anchor body is removably attached to an outer actuation element.

Example 36. The implantable device of Example 32, wherein the actuation element is disposed radially inward of the delivery catheter.

Example 37. The implantable device of Example 35, wherein movement of the inner actuation element can move the inner anchor body between a closed position and an open position.

Example 38. The implantable device of Example 35, wherein movement of the outer actuation element can move the outer anchor body between a closed position and an open position.

Example 39. The implantable device of any one of Examples 20-38, wherein the actuation element is connected to the anchor portion such that a user can provide a tensioning force to the actuation element to cause the anchor portion to move from an expanded position having an expanded width to a narrowed position having a narrowed width, wherein the expanded width is greater than the narrowed width.

Example 40. A method of repairing a native valve comprising:
positioning anchor portions such that leaflets of a native heart valve are disposed in the anchors; and
drawing the anchors and portions of the leaflets into an open end of a capture element having a first end, a second end and a cavity between the first end and the second end.

Example 41. The method of Example 40 further wherein the anchor portion is extendable out of the cavity of the capture element and retractable into the cavity of the capture element;

Example 42. The method of any of Examples 40-41 further comprising blocking blood flow with the capture element.

Example 43. The method of any of Examples 40-41 further comprising decoupling the anchors and capture element from a delivery catheter.

Example 44. An implantable device comprising: (i) an anchor portion comprising one or more anchors, wherein the anchor portion coupled with the capture element, and (ii) a rotating member coupled with the one or more anchors, wherein the rotating member is configured to draw native valve leaflet tissue into the one or more anchors.

Example 45. The implantable device of Example 44, wherein the rotating member is cylindrical.

Example 46. The implantable device of any one of Examples 44-45, wherein the rotating member comprises one or more projections, notches, or other gripping members spaced throughout the rotating member for coupling with leaflets.

Example 47. The implantable device of any one of Examples 44-46, wherein the rotating member comprises a ridge that is threaded along a surface of the rotating member.

Example 48. The implantable device of any one of Examples 44-47, wherein in response to the rotating member rotating in a first direction, the rotating member draws a leaflet into the one or more anchors and wherein in response to the rotating member rotating in a second direction, the rotating member moves the leaflet out of the one or more anchors.

Example 49. The implantable device of any one of Examples 44-48, wherein the rotating member is removably attached to an actuation element.

Example 50. A device adapted to be implanted between leaflets of a native heart valve, the device comprising: (i) a gripping member including a base arm and a moveable arm, the gripping member configured to move between an open position and a closed position, wherein in the closed position, the gripping member is configured to grasp a leaflet of a native heart valve between the base arm and the movable arm; and (ii) a leaflet repositioning device configured to reposition the leaflet relative to the base arm while the gripping member is in the closed position.

Example 51. The device of Example 50, wherein the leaflet repositioning device is configured to move the movable arm, relative to the base arm, toward a centerline of the device while the gripping member is in the closed position.

Example 52. The device of Example 51, wherein the movable arm has a proximal end and a distal end, and wherein the Example leaflet repositioning device includes a retraction element attached to the proximal end, and wherein pulling the retraction element while the gripping member is in the closed position causes the distal end to move toward a centerline of the device.

Example 53. The device of Example 52, wherein the device further comprises a coaptation element positioned along the centerline of the device, wherein pulling the retraction element causes the movable arm to retract into the coaptation element.

Example 54. The device of any of Examples 51-53, further comprising a lock configured to secure the movable arm in position after being moved by the leaflet repositioning device.

Example 55. The device of any of Examples 50-54, further comprising a securing element disposed on the movable arm for engaging the leaflet.

Example 56. The device of Example 55, wherein the securing element includes one or more barbs.

Example 57. The device of Example 55, further comprising a second securing element disposed on the base arm for engaging the leaflet.

Example 58. The device of Example 50, further comprising a securing element disposed on the movable arm for engaging the leaflet wherein the leaflet repositioning device is configured to move the securing element, relative to the movable arm, toward a centerline of the device while the gripping member is in the closed position.

Example 59. The device of Example 58, wherein the movable arm has a proximal end and a distal end, and wherein the leaflet repositioning device includes a retraction element attached to the securing element end, and wherein pulling the retraction element while the gripping member is in the closed position causes the securing element to move toward a centerline of the device.

Example 60. The device of Example 59, wherein the device further comprises a coaptation element positioned along the centerline of the device.

Example 61. The device of any of Examples 58-60, further comprising a lock configured to secure the securing element in position relative to the movable arm after being moved by the leaflet repositioning device.

Example 62. The device of any of Examples 58-61, wherein the securing element includes one or more barbs.

Example 63. The device of any of Examples 58-62, wherein the securing element has a distal position and a proximal position that is inward of the distal position, and wherein the leaflet repositioning device is configured to move the securing element repeatedly between the distal position and proximal position to incrementally move the leaflet toward the centerline of the device.

Example 64. The device of Example 63, wherein the leaflet repositioning device includes a ratcheting device operable to move the securing element repeatedly between the distal position and proximal position.

Example 65. The device of Example 63 or 64, further comprising a second securing element disposed on the base arm for engaging the leaflet, and wherein when the securing element disposed on the movable arm moves from the proximal position to the distal position, the second securing element holds the leaflet in position relative to the movable arm.

Example 66. The device of Example 50, wherein the leaflet repositioning device is configured to rotate the movable arm, relative to the base arm, about a longitudinal axis of the movable arm while the gripping member is in the closed position.

Example 67. The device of Example 66, wherein rotating the movable arm moves the leaflet relative to the base arm.

Example 68. The device of any of Examples 66-67, further comprising a lock configured to secure the movable arm in rotational position after being moved by the leaflet repositioning device.

Example 69. The device of any of Example 66-68, further comprising a securing element disposed on the movable arm for engaging the leaflet.

Example 70. The device of Example 69, wherein the securing element includes one or more barbs.

Example 71. The device of Example 66, further comprising a second gripping member including a second base arm and a second moveable arm, the second gripping member configured to move between an open position and a closed position, wherein in the closed position, the second gripping member is configured to grasp a second leaflet of a native heart valve between the second base arm and the second movable arm.

Example 72. The device of Example 71, wherein the leaflet repositioning device is configured to rotate the second movable arm, relative to the second base arm, about a second longitudinal axis of the second movable arm while the second gripping member is in the closed position.

Example 73. The device of Example 71, further comprising a second leaflet repositioning device, wherein the second repositioning device is configured to rotate the second movable arm, relative to the second base arm, about a second longitudinal axis of the second movable arm while the second gripping member is in the closed position.

Example 74. The device of any of Examples 71-73, wherein the second movable arm and the movable arm are configured to rotate simultaneously in opposite directions.

Example 75. A delivery system for a device adapted to be implanted between leaflets of a native heart valve, comprising: (i) an implant catheter assembly having a sheath with a distal end portion comprising a capture mechanism for releasably attaching the sheath to the device, and (ii) the implantable device of any of Examples 50-57.

Example 76. A method of repairing a native valve comprising, the method comprising: (i) delivering an implantable device to the native valve; (ii) positioning the implantable device in annulus of the native valve; (iii) closing a gripping member of the implantable device to grasp a leaflet of the native valve, wherein the gripping member has a base arm and a movable arm; and (iv) repositioning the leaflet relative to the base arm while the gripping member remains closed.

Example 77. The method of Example 76, wherein repositioning the leaflet further comprises moving the movable arm, relative to the base arm, toward a centerline of the device.

Example 78. The method of Example 77, moving the movable arm further comprises applying tension to a retraction element coupled to the movable arm.

Example 79. The method of Example 78, wherein applying tension to the retraction element further comprises retracting the movable arm into a coaptation element of the device.

Example 80. The method of any of Examples 76-79, further comprising locking the movable arm in position after repositioning the leaflet.

Example 81. The method of any of Example 76-80, wherein closing the gripping member further comprises engaging the leaflet with a securing element disposed on the movable arm.

Example 82. The method of Example 81, wherein the securing element includes one or more barbs.

Example 83. The method of Example 81 or 82, wherein closing the gripping member further comprises engaging the leaflet with a second securing element disposed on the base arm.

Example 84. The method of Example 76, wherein a securing element is disposed on the movable arm, and wherein repositioning the leaflet further comprises moving the securing element, relative to the movable arm, toward a centerline of the device while the gripping member is in the closed position.

Example 85. The method of Example 84, wherein moving the securing element further comprises applying tension to a retraction element coupled to the securing element.

Example 86. The method of any of Examples 84-85, further comprising locking the securing element in position relative to the movable arm after being moved by the leaflet repositioning device.

Example 87. The method of any of Examples 84-86, wherein the securing element includes one or more barbs.

Example 88. The method of any of Examples 84-87, wherein the securing element has a distal position and a proximal position that is inward of the distal position, and wherein repositioning the leaflet further comprises repeatedly moving the securing element between the distal position and proximal position to incrementally move the leaflet toward the centerline of the device.

Example 89. The method of Example 88, wherein a second securing element is disposed on the base arm for engaging the leaflet, and wherein repeatedly moving the securing element between the distal position and proximal position further comprises holding the leaflet in position relative to the movable arm with the second securing element when the securing element disposed on the movable arm moves from the proximal position to the distal position.

Example 90. The method of Example 76, wherein repositioning the leaflet further comprises rotating the movable arm, relative to the base arm, about a longitudinal axis of the movable arm while the gripping member is in the closed position.

Example 91. The method of Example 90, wherein rotating the movable arm moves the leaflet relative to the base arm.

Example 92. The method of Example 90 or 91, further comprising locking the movable arm in a rotational position after repositioning the leaflet.

Example 93. The method of any of Examples 90-91, wherein closing the gripping member further comprises engaging the leaflet with a securing element disposed on the movable arm.

Example 94. The method of Example 93, wherein the securing element includes one or more barbs.

Example 95. The method of Example 76, further comprising closing a second gripping member of the implantable device to grasp a second leaflet of the native valve, wherein the second gripping member has a second base arm and a second movable arm, and repositioning the second leaflet relative to the second base arm while the second gripping member remains closed.

Example 96. The method of Example 95, wherein repositioning the second leaflet further comprises rotating the second movable arm, relative to the second base arm, about a second longitudinal axis of the second movable arm while the second gripping member is in the closed position.

Example 97. The method of Example 96, rotating the second movable arm and rotating the first movable arm are done simultaneously in opposite directions.

Example 98. The method of any of Examples 76-97, further comprising sterilizing the implantable device.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. An implantable device comprising:
a capture element having a first end, a second end and an inner cavity between the first end and the second end;
an anchor portion comprising one or more anchors, wherein the anchor portion is disposed at least partially in the capture element, wherein the one or more anchors are configured to capture one or more leaflets of a native heart valve;
wherein the anchor portion is extendable out of the inner cavity of the capture element and retractable into the inner cavity of the capture element; and
wherein the capture element and the anchor portion are configured to draw native valve leaflet tissue into the inner cavity when the anchor portion is retracted into the inner cavity of the capture element.

2. The implantable device of claim 1, wherein the inner cavity extends from the first end of the capture element to the second end of the capture element.

3. The implantable device of claim 2, wherein in a closed position, the anchor portion is housed entirely within the inner cavity.

4. The implantable device of claim 2, wherein in an open position, the anchor portion is housed at least partially outside of the inner cavity.

5. The implantable device of claim 1, wherein the capture element comprises an end wall at the first end of the capture element, the end wall comprising an opening.

6. The implantable device of claim 1, wherein the second end of the capture element is open, such that the anchor portion can be moved in and out of the capture element from the second end.

7. An implantable device comprising:
an anchor portion comprising one or more anchors, wherein the anchor portion is coupled to a capture element,
a rotating member coupled to the anchor portion; and
wherein the rotating member is configured to draw native valve leaflet tissue into the one or more anchors.

8. The implantable device of claim 7, wherein the rotating member comprises one or more projections, notches, or gripping members spaced throughout the rotating member for coupling with leaflets.

9. A device adapted to be implanted between leaflets of a native heart valve, the device comprising:
a gripping member including a base arm and a moveable arm, the gripping member configured to move between an open position and a closed position, wherein in the closed position, the gripping member is configured to grasp a leaflet of the native heart valve between the base arm and the moveable arm; and
a leaflet repositioning device configured to reposition the leaflet relative to the base arm while the gripping member is in the closed position.

10. The device of claim 9, wherein the leaflet repositioning device is configured to move the moveable arm, relative to the base arm, toward a centerline of the device while the gripping member is in the closed position.

11. The device of claim 10, wherein the moveable arm has a proximal end and a distal end, and wherein the leaflet repositioning device includes a retraction element attached to the proximal end, and wherein pulling the retraction element while the gripping member is in the closed position causes the distal end to move toward the centerline of the device.

12. The device of claim 11, wherein the device further comprises a coaptation element positioned along the centerline of the device, wherein pulling the retraction element causes the moveable arm to retract into the coaptation element.

13. The device of claim 9, further comprising a lock configured to secure the moveable arm in position after being moved by the leaflet repositioning device.

14. The device of claim 9, further comprising a securing element disposed on the moveable arm for engaging the leaflet.

15. The device of claim 14, further comprising a lock configured to secure the securing element in position relative to the moveable arm after being moved by the leaflet repositioning device.

16. The device of claim 14, wherein the securing element has a distal position and a proximal position that is inward of the distal position, and wherein the leaflet repositioning device is configured to move the securing element repeatedly between the distal position and the proximal position to incrementally move the leaflet toward a centerline of the device.

17. The device of claim 16, wherein the leaflet repositioning device includes a ratcheting device operable to move the securing element repeatedly between the distal position and the proximal position.

18. The device of claim 9, further comprising a securing element disposed on the moveable arm for engaging the leaflet wherein the leaflet repositioning device is configured to move the securing element, relative to the moveable arm, toward a centerline of the device while the gripping member is in the closed position.

19. The device of claim 18, wherein the moveable arm has a proximal end and a distal end, and wherein the leaflet repositioning device includes a retraction element attached to an end of the securing element, and wherein pulling the retraction element while the gripping member is in the closed position causes the securing element to move toward the centerline of the device.

20. The device of claim 19, wherein the device further comprises a coaptation element positioned along the centerline of the device.
